(19) United States
(12) Patent Application Publication
(10) Pub. No.: US 2024/0111954 A1
(43) Pub. Date: Apr. 4, 2024

(54) EVIDENCE NETWORK NAVIGATION

(71) Applicant: Scinapsis Analytics Inc., dba BenchSci, Toronto (CA)

(72) Inventors: Craig Farley NEWELL, Toronto (CA); Tom LEUNG, North York (CA); Elvis WIANDA, Oakville (CA); Amit BRONNER, Toronto (CA); Christian BATTISTA, Hamilton (CA)

(73) Assignee: Scinapsis Analytics Inc., dba BenchSci, Toronto (CA)

(21) Appl. No.: 17/958,217

(22) Filed: Sep. 30, 2022

(51) Int. Cl. G06F 40/289; G06F 40/30; G06V 30/14; G16H 70/00

(52) U.S. Cl. CPC G06F 40/289; G06F 40/30; G06V 30/1456; G16H 70/00

(57) ABSTRACT

A method implements evidence network navigation. The method includes receiving a user input corresponding to an entity of an ontology library; and generating an evidence graph using the user input, wherein the evidence graph includes an evidence node representing the entity from the ontology library and includes an evidence edge representing a file that includes the entity in a result graph, and wherein the result graph includes a result node representing the entity and a result edge representing a semantic relationship of the result node in a sentence from the file. The method further includes presenting the evidence graph.

### BACKGROUND

Biomedical information includes literature and writings that describe evidence from experiments and research of biomedical science that provides the basis for modern medical treatments. Biomedical information is published in publications in physical or electronic form and may be distributed in electronic form using files. Databases of biomedical information provide access to the electronic forms of the publications. A challenge is for computing systems to navigate through the evidence provided by databases of biomedical information.

### SUMMARY

In general, in one or more aspects, the disclosure relates to a method implementing evidence network navigation. The method includes receiving a user input corresponding to an entity of an ontology library; and generating an evidence graph using the user input, wherein the evidence graph includes an evidence node representing the entity from the ontology library and includes an evidence edge representing a file that includes the entity in a result graph, and wherein the result graph includes a result node representing the entity and a result edge representing a semantic relationship of the result node in a sentence from the file. The method further includes presenting the evidence graph.

In general, in one or more aspects, the disclosure relates to a system implementing evidence network navigation. The system includes an evidence graph controller configured to generate an evidence graph and an application executing on one or more servers. The application is configured for receiving a user input corresponding to an entity of an ontology library, generating the evidence graph using the user input, wherein the evidence graph includes an evidence node representing the entity from the ontology library and includes an evidence edge representing a file that includes the entity in a result graph, and wherein the result graph includes a result node representing the entity and a result edge representing a semantic relationship of the result node in a sentence from the file. The system is further configured for presenting the evidence graph.

In general, in one or more aspects, the disclosure relates to a method of evidence network navigation. The method includes transmitting a request; and displaying an evidence graph received in a response to the request, wherein the evidence graph includes an evidence node representing an entity from an ontology library and includes an evidence edge representing a file that includes the entity in a result graph, and wherein the result graph includes a result node representing the entity and a result edge representing a semantic relationship of the result node in a sentence from the file.

Other aspects of the invention will be apparent from the following description and the appended claims.

### BRIEF DESCRIPTION OF DRAWINGS

FIG. 1A, FIG. 1B, FIG. 1C, FIG. 1D, and FIG. 1E show diagrams of systems in accordance with disclosed embodiments.

FIG. 2 shows a flowchart in accordance with disclosed embodiments.

FIG. 3A, FIG. 3B, FIG. 4, FIG. 5, FIG. 6, FIG. 7A, FIG. 7B, FIG. 7C, FIG. 7D, FIG. 7E, FIG. 7F, FIG. 7G, FIG. 7H, FIG. 8A, FIG. 8B, FIG. 8C, FIG. 8D, FIG. 9A, FIG. 9B, FIG. 9C, FIG. 10A, FIG. 10B, FIG. 11A, FIG. 11B, FIG. 11C, FIG. 11D, FIG. 11E, FIG. 11F, and FIG. 11G show examples in accordance with disclosed embodiments.

FIG. 12A and FIG. 12B show computing systems in accordance with disclosed embodiments.

### DETAILED DESCRIPTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as by the use of the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In general, embodiments of the disclosure implement evidence network navigation. An evidence network may be implemented as an evidence graph generated from evidence result networks. The nodes of the evidence graph represent entities (proteins, diseases, experimentation techniques, etc.) of an ontology library stored to a repository. The edges of the evidence graph represent the files (including publications, patents, electric lab notebook (ELN) data, summary documents, etc.) that record evidence of biomedical experiments related to the entities of the nodes of the edges. For example, a node may represent a protein (e.g., EPAS1) and another node may represent a disease (e.g., cancer). A file may include evidence describing an experiment with a sentence that includes both of the entities (e.g., “ . . . EPAS1 may increase expression of other proteins in cancer cells . . . ”). The edge between the two nodes for EPAS1 and cancer would include the file.

A user can click on the edges and the nodes to further explore the biomedical information. The evidence graph may be expanded by clicking on a node. The evidence graph may be filtered based on nodes or edges. Filters for nodes may be based on entities (e.g., specific names or aliases), entity types, etc. Filters for edges may be based on types of files, types of publications, publication dates, number of experiments or publications, etc.

In general, embodiments of the disclosure implement evidence result networks. An evidence result network may be implemented as a graph generated from a file or other scientific evidence by a computing system. The system receives a file (i.e., including but not limited to publications of biomedical information, patents, documents of experimental results originating from external and internal customer sources, etc.) that stores the evidence using text, images, data, etc. The evidence (i.e., the data of a file describing scientific results) is processed to identify the sentences and images in the file. The file may be referred to as a source of evidence of a scientific result. One or more machine learning models may be used to process the sentences and images to generate graphs (evidence result networks) that represent the evidence demonstrated from experiments described by the sentences and images from the file. An ontology library (saved as a collection of data records) is used to identify terms and phrases from the text and images of the file that relate to entities with biomedical meaning. For example, the ontology library may store the names of proteins, diseases, experimentation techniques, etc. The entities from the ontology library may be recognized during the processing of the file to preserve the meaning of terms and phrases from the text and images in the graphs generated by the system.

The machine learning models used by the system may be trained to understand evidence both written and visual. For example, a machine learning model may be trained to recognize and tag entities in biomedical information, defined by the data records of the ontology library, that appear in a sentence. Additional machine learning models (semantic tree generators, image recognizers, etc.) may be trained with biomedical data (text and images) to be customized for biomedical data.

After a file is processed to generate a set of result graphs for the evidence described by the data of the file, the graphs and images from the file may be displayed to a user. For example, a user interested in the relationship between two entities (e.g., a protein and a disease) may locate a file corresponding to a biomedical publication that includes the two entities in a graph generated from a sentence or image from the file. Graphs and images that describe the relationships between the entities may then be displayed to the user.

The figures show diagrams of embodiments that are in accordance with the disclosure. The embodiments of the figures may be combined and may include or be included within the features and embodiments described in the other figures of the application. The features and elements of the figures are, individually and as a combination, improvements to the technology of biomedical information processing and machine learning models. The various elements, systems, components, and steps shown in the figures may be omitted, repeated, combined, and/or altered as shown from the figures. Accordingly, the scope of the present disclosure should not be considered limited to the specific arrangements shown in the figures.

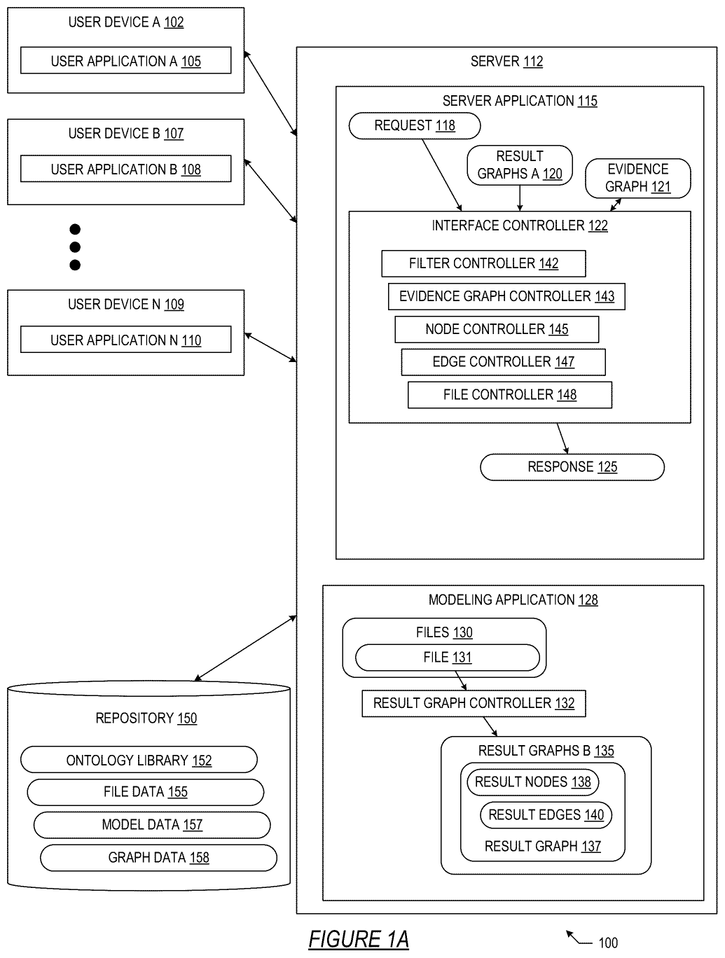

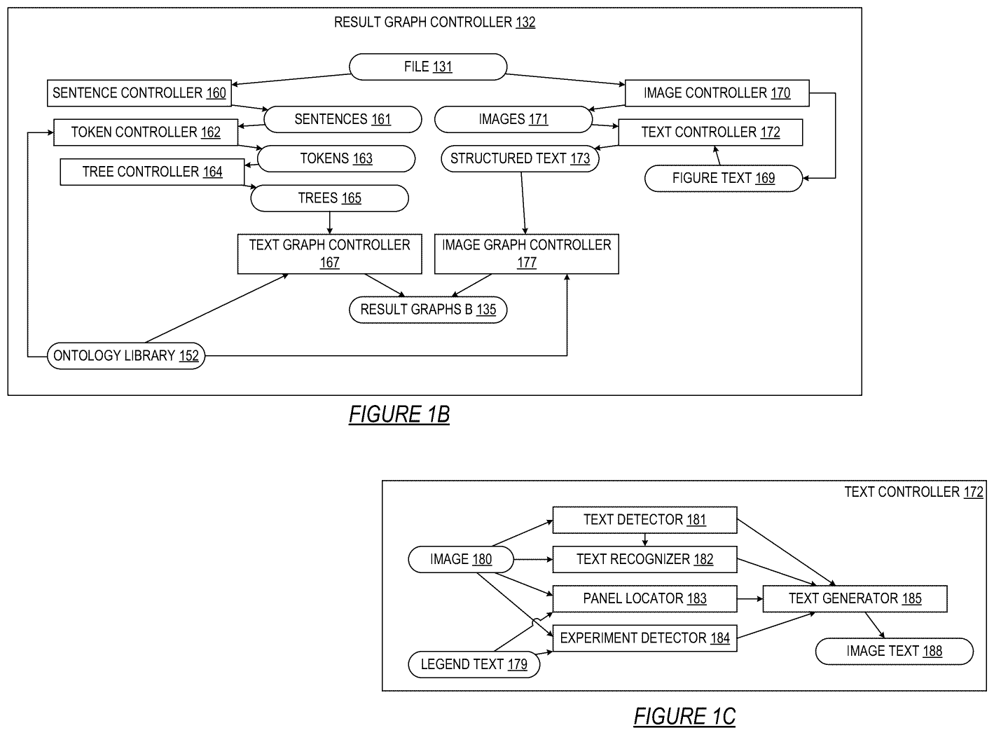

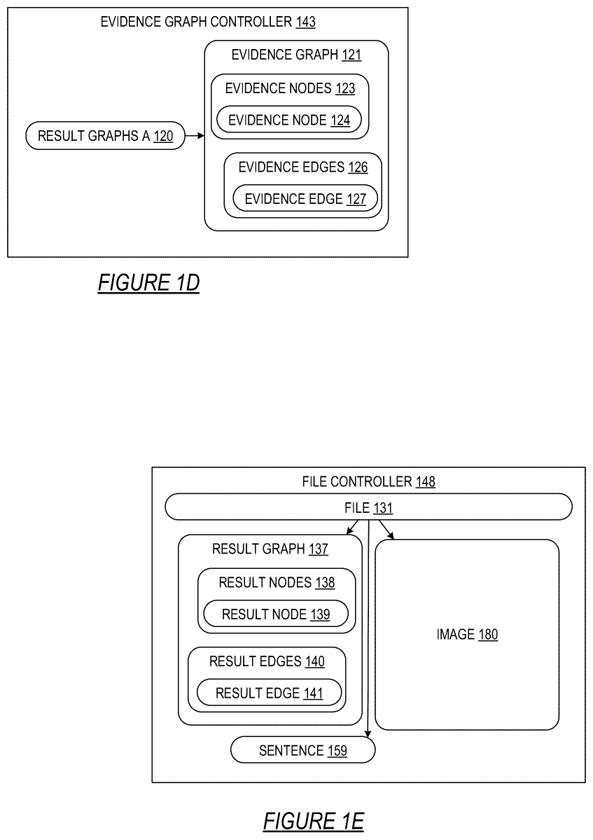

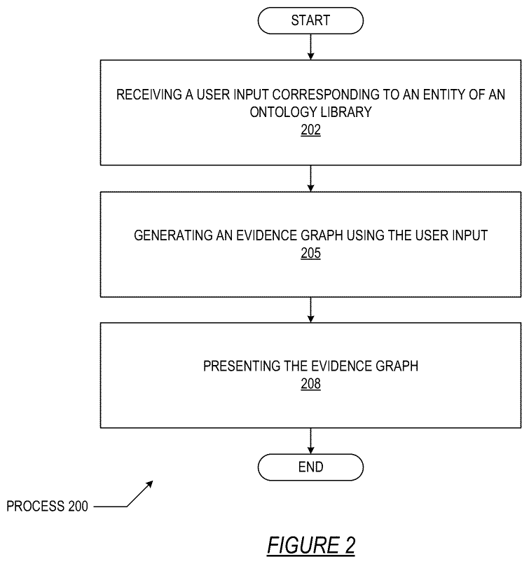

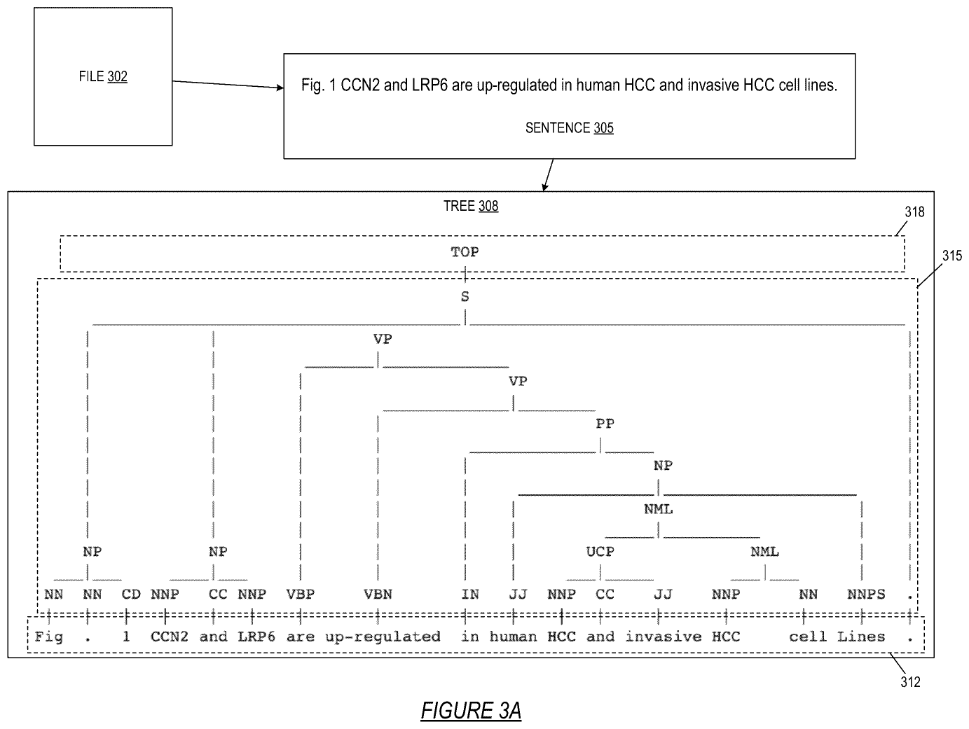

Turning to FIG. 1A, the system (100) implements evidence network navigation by converting biomedical information from files to result graphs and generating an evidence graph from the result graphs. The system (100) receives requests (e.g., the request (118)) and generates responses (e.g., the response (125)) using the result graphs A (120). The system (100) generates the result graphs A (120) from biomedical information (e.g., the files (131)) stored in the file data (155) using multiple machine learning and natural language processing models. The system (100) uses the result graphs A (120) to generate the evidence graph (121). The system (100) generates the response (125) using the evidence graph (121). The system (100) may display the evidence graph (121), the result graphs A (120), and the images from the files of the file data (155) to users operating the user devices A (102) and B (107) through N (109). The system (100) includes the user devices A (102) and B (107) through N (109), the server (112), and the repository (150).

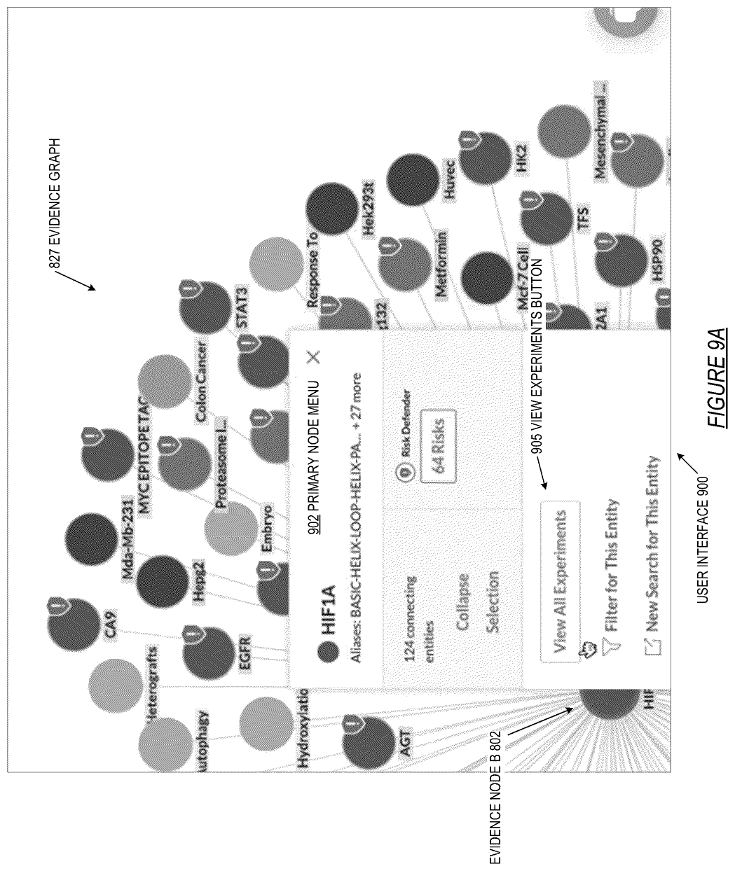

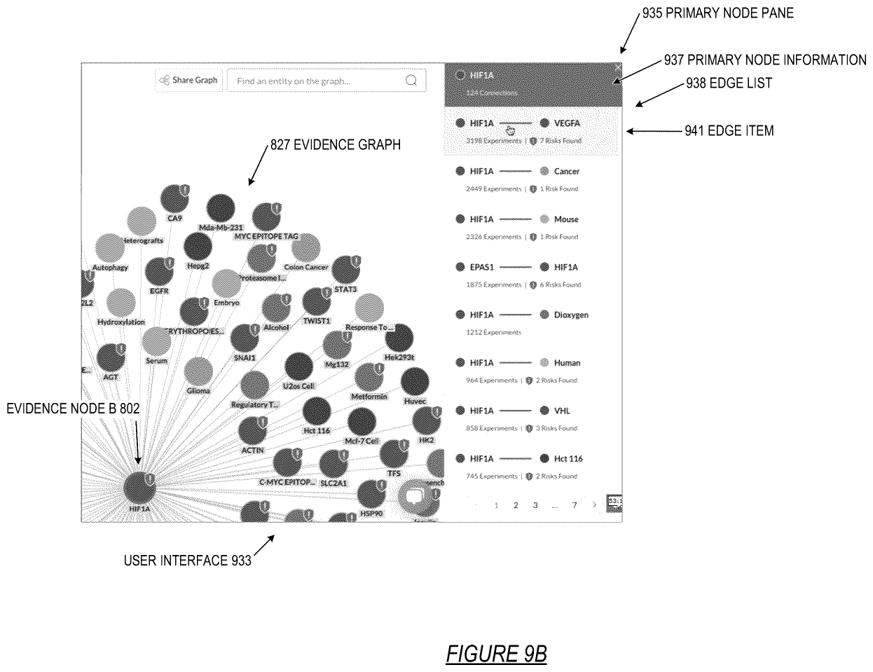

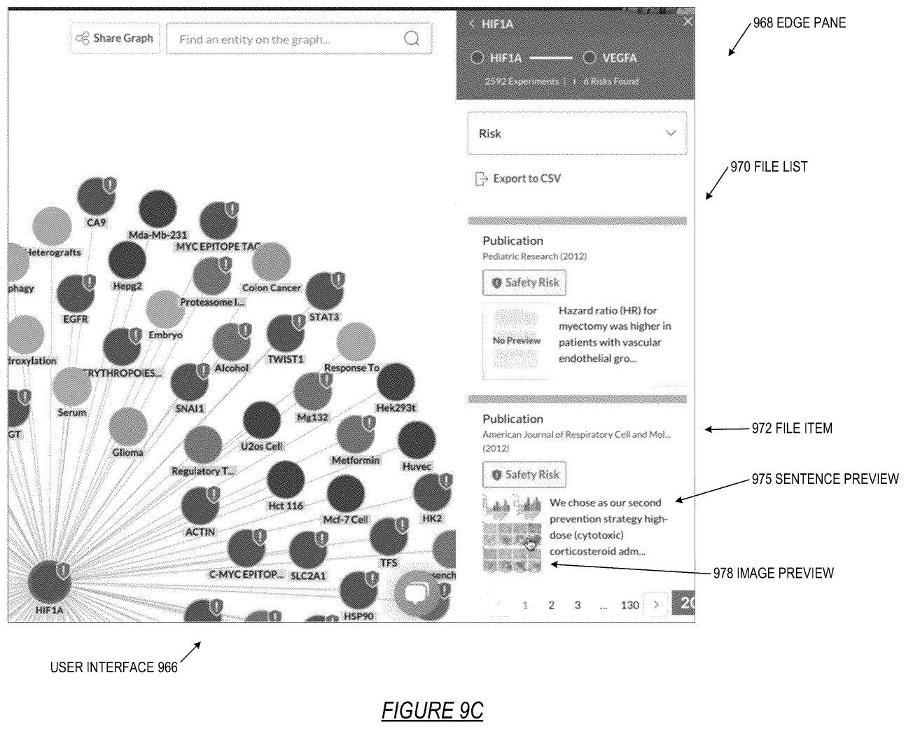

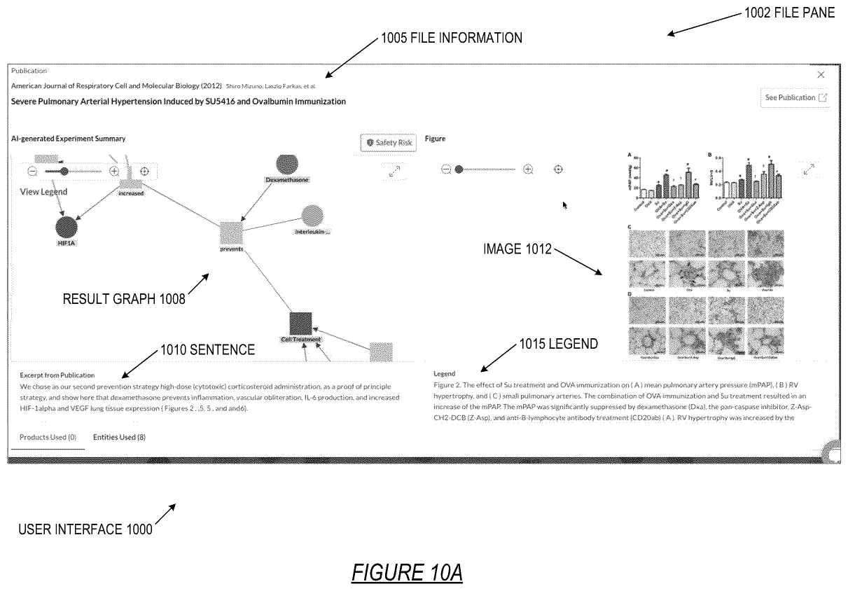

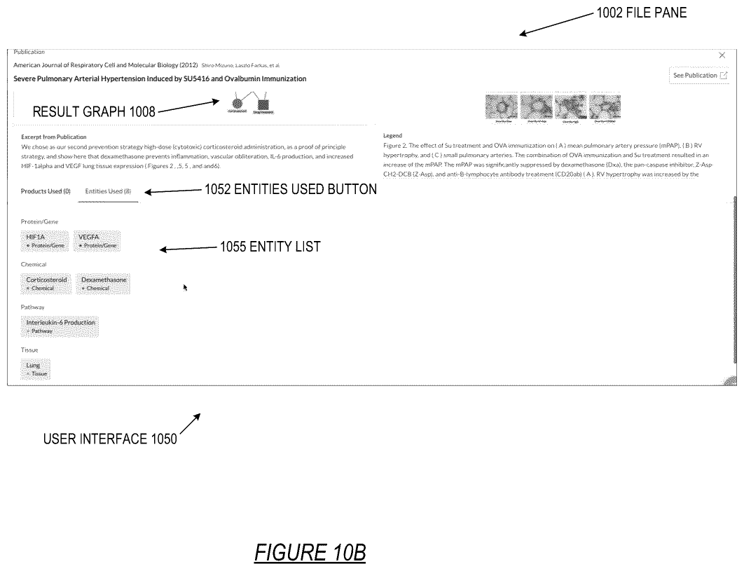

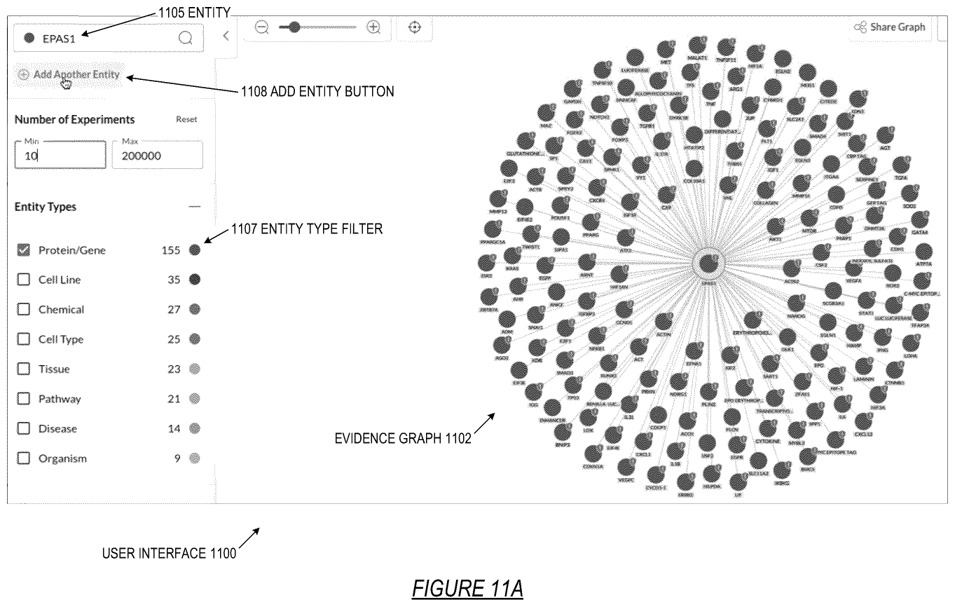

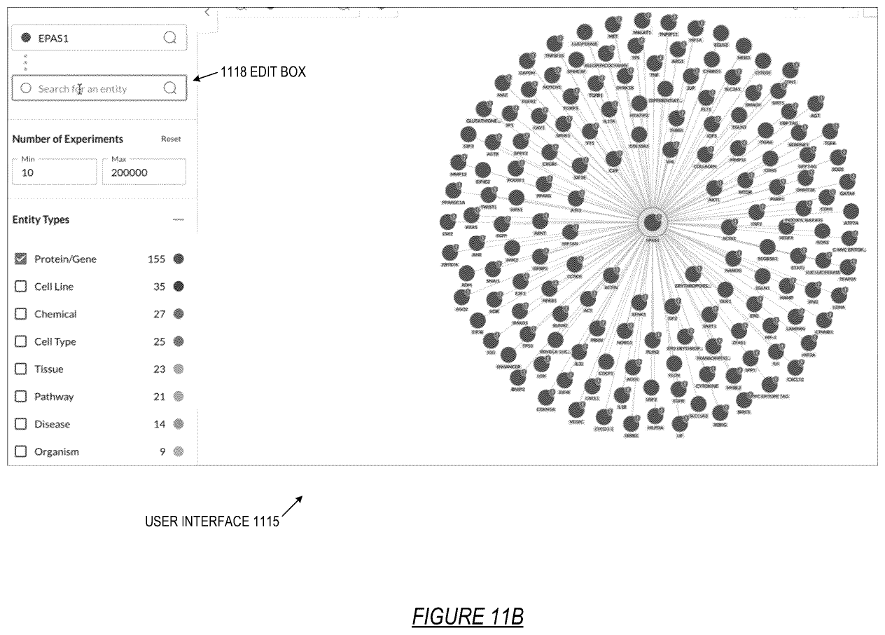

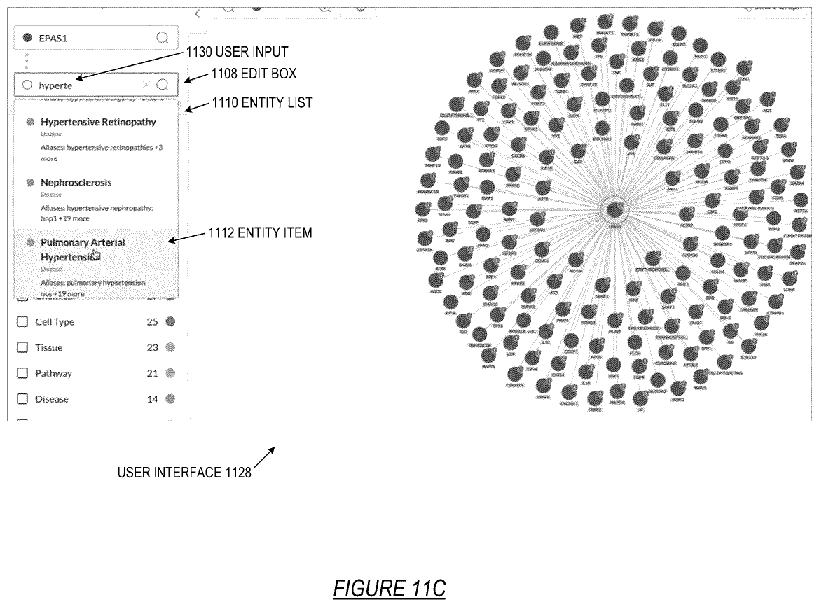

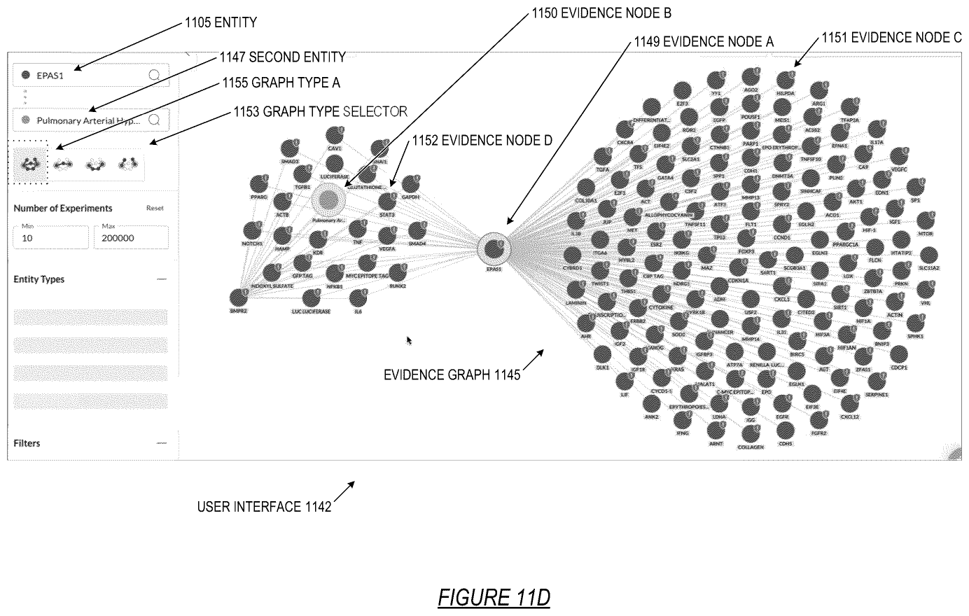

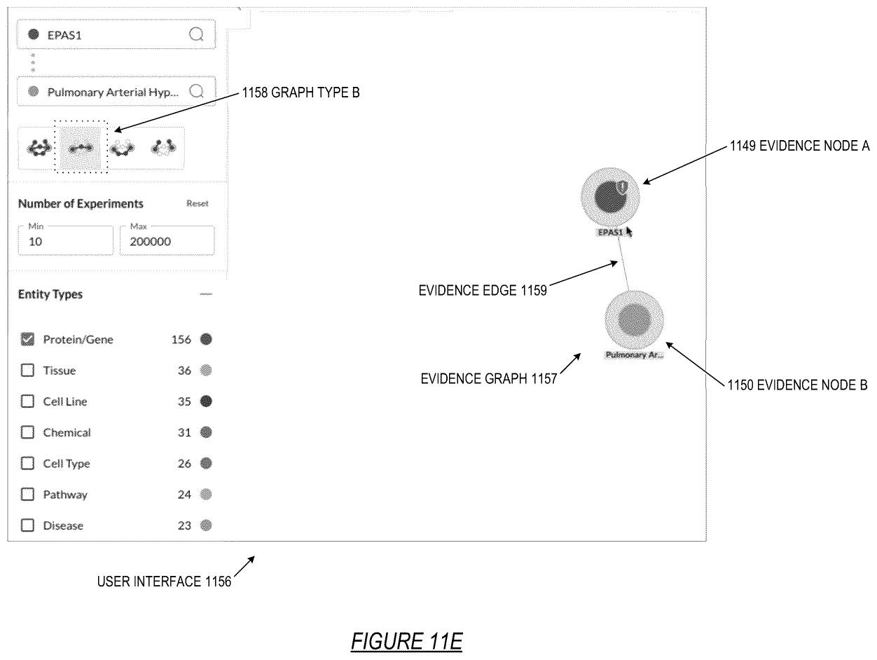

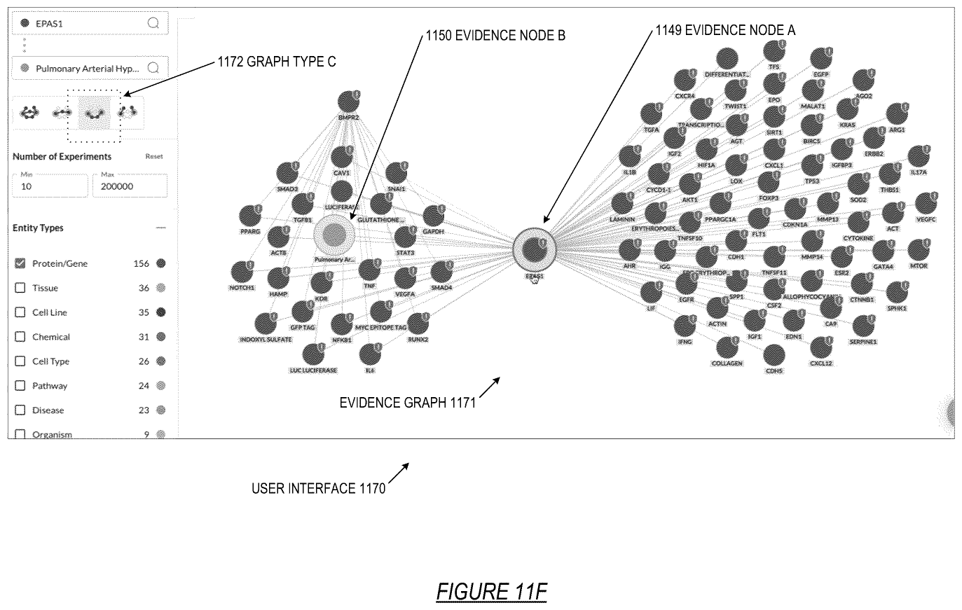

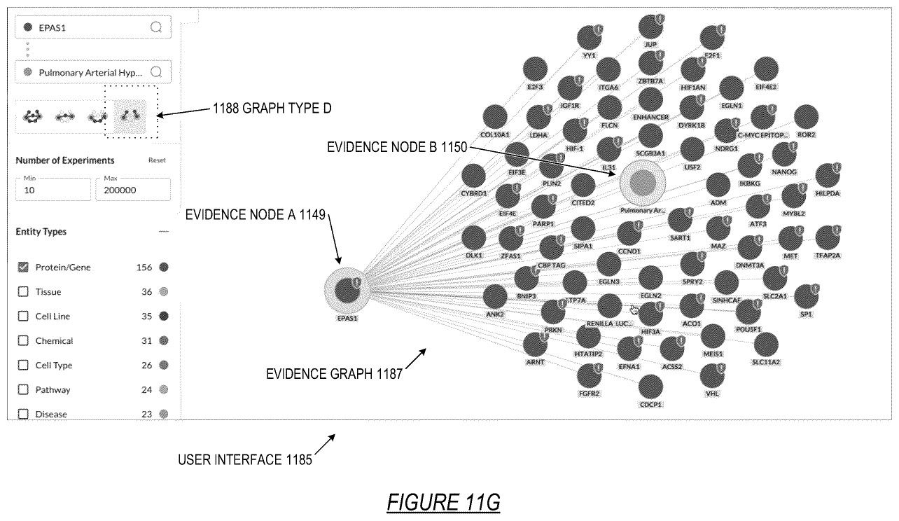

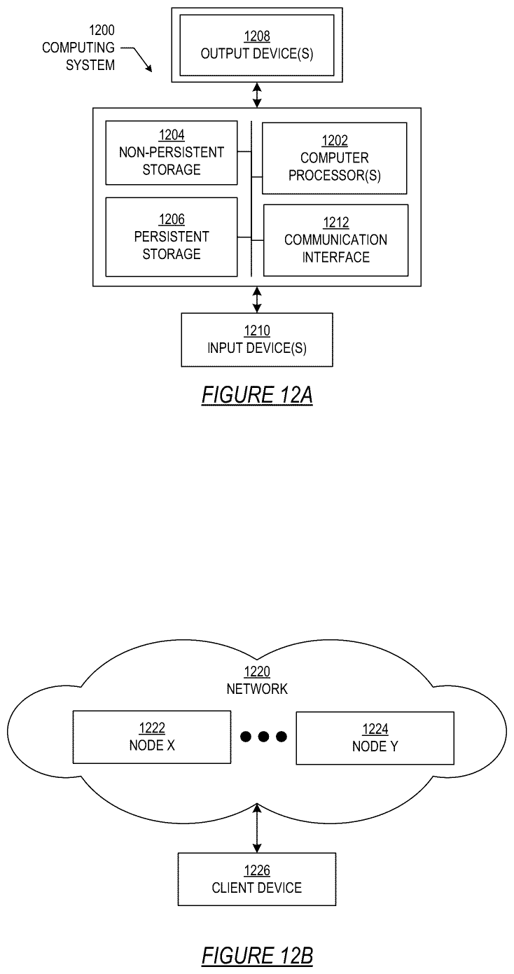

The server (112) is a computing system (further described in FIG. 12A). The server (112) may include multiple physical and virtual computing systems that form part of a cloud computing environment. In one embodiment, execution of the programs and applications of the server (112) is distributed to multiple physical and virtual computing systems in the cloud computing environment. The server (112) includes the server application (115) and the modeling application (128).

The server application (115) is a collection of programs that may execute on multiple servers of a cloud environment, including the server (112). The server application (115) receives the request (118) and generates the response (125) based on the result graphs A (120) using the evidence graph (121) and the interface controller (122). The server application (115) may host websites accessed by users of the user devices A (102) and B (107) through N (109) to view information from the evidence graph (121), the result graphs A (120), and the file data (155). The websites hosted by the server application (115) may serve structured documents (hypertext markup language (HTML) pages, extensible markup language (XML) pages, JavaScript object notation (JSON) files and messages, etc.). The server application (115) includes the interface controller (122), which processes the request (118) using the result graphs A (120) and the evidence graph (121).

The request (118) is a request from one of the user devices A (102) and B (107) through N (109). In one embodiment, the request (118) is a request for information about one or more entities defined in the ontology library (152), described in the file data (155), and graphed in the graph data (158). In one embodiment, the request (118) may specify additional filters for the list of entities. The structured text below (formatted in accordance with JSON) provides an example of entities that may be specified in the request (118) using key value pairs.

```
{
  “entity type”: “protein”,
  “entity”: “BRD9”,
}
{
  “entity type”: “disease”,
  “entity”: “breast cancer”,
}
```

The result graphs A (120) are generated with the modeling application (128), described further below. The result graphs A (120) includes nodes and edges in which the nodes correspond to text from the file data (155) and the edges correspond to semantic relationships between the nodes. The result graphs A (120) are directed graphs in which the edges identify a direction from one node to a subsequent node in the result graphs A (120). In one embodiment, the result graphs A (120) are acyclic graphs. The result graphs A (120) may be stored in the graph data (158) of the repository (150).

The evidence graph (121) is generated from the result graphs A (120) and may be stored in the graph data (158). Nodes of the evidence graph (121) represent entities from the ontology library (152). The edges of the evidence graph (121) identify files (e.g., publications) that record evidence of experiments related to the entities represented by the nodes of the evidence graph (121). For example, an edge may represent a file of a publication with a sentence (graphed as one of the result graphs A (120)) that includes the entities corresponding to the nodes of the edge. The edges of the evidence graph (121) may generate using the result graphs A (120). The evidence graph (121) may be stored in the graph data (158) of the repository (150).

The interface controller (122) is a collection of programs that may operate on the server (112). The interface controller (122) processes the request (118) using the result graphs A (120) and the evidence graph (121) to generate the response (125). In one embodiment, the interface controller (122) searches the graph data (158) to identify the result graphs A (120) (which may include some of the result graphs from the result graphs B (135)) that include information about the entities identified in the request (118). The interface controller (122) may update the evidence graph (121) using information from the request (118). For example, the request (118) may include filters to apply to the evidence graph (121), instructions to expand a node of the evidence graph (121), etc.

The filter controller (142) is a collection of programs that may operate on the server (112). The filter controller (142) processes the request (118) to apply filters to the evidence graph (121). The filters may filter the nodes and edges of the evidence graph (121). For example, the nodes may be filtered by entity names and entity types. The edges may be filtered by the number of experiments (e.g., the number of files with evidence including the entity of a node) sufficient to have an edge displayed between nodes of the evidence graph (121). A minimum number of experiments or files, a maximum number of experiments or files, etc., may be used as the filter. The edges may also be filtered by the type of publication (“published”, “preprint”, etc.) of the evidence in the file. In one embodiment, the filter controller (142) may process instructions from the request (118) that are generated responsive to a panel displayed on a left side of a user interface of one of the user application A (105) and user application B (108) through user application N (110).

The evidence graph controller (143) is a collection of programs that may operate on the server (112). The evidence graph controller (143) presents the evidence graph (121) in response to updates to the evidence graph (121). In one embodiment, the evidence graph controller (143) may generate HTML code that is transmitted to one of the user devices A (102) and B (107) through N (109), which display the evidence graph (121).

The node controller (145) is a collection of programs that may operate on the server (112). The node controller (145) updates the evidence graph (121) based on selections of a node from the request (118). In one embodiment, the node controller (145) may process instructions from the request (118) that are generated responsive to a popup menu displayed upon selection of a node from the evidence graph (121) using a user interface of one of the user applications A (105) and B (108) through N (110).

For example, the request (118) may include instructions to expand a node of the evidence graph (121) in response to a selection of a popup menu displayed on a node. The node controller (145) may add additional nodes to the evidence graph (121) to expand the selected node. The additional nodes may be located by searching the result graphs A (120) for graphs that include the entity represented by the selected node.

The edge controller (147) is a collection of programs that may operate on the server (112). The edge controller (147) may present summaries of information corresponding to one or more files based on a selection of an edge from the evidence graph (121). In one embodiment, the edge controller (147) may process instructions from the request (118) that are generated responsive to a panel displayed on a right side of a user interface of one of the user applications A (105) and B (108) through N (110).

The file controller (148) is a collection of programs that may operate on the server (112). In one embodiment, the file controller (148) may present information from a file (sentence, result graph, image, etc.).

The response (125) is generated by the interface controller (122) in response to the request (118) using the result graphs A (120). In one embodiment, the response (125) includes the evidence graph (121), which may be updated based on the request (118). The response (125) may further include one or more of the result graphs A (120) and information from the file data (155). Portions of the response (125) may be displayed by the user devices A (102) and B (107) through N (109) that receive the response (125).

The modeling application (128) is a collection of programs that may operate on the server (112). The modeling application (128) generates the result graphs B (135) from the files (130) using a result graph controller (132).

The files (130) include biomedical information and form the basis for the result graphs B (135). The files (130) include the file (131), which is the basis for the result graph (137). Each file includes multiple sentences and may include multiple images of evidence. The evidence may identify how different entities, defined in the ontology library (152), affect each other. For example, entities that are proteins may suppress or enhance the expression of other entities and affect the prevalence of certain diseases. Types of entities include proteins, genes, diseases, experiment techniques, chemicals, cell lines, pathways, tissues, cell types, organisms, etc. In one embodiment, nouns and verbs from the sentences of the file (131) are mapped to the result nodes (138) of the result graph (137). In one embodiment, the semantic relationships between the words in the sentences corresponding to the result nodes (138) are mapped to the result edges (140). In one embodiment, one file serves as the basis for multiple result graphs. In one embodiment, one sentence from a file may serve as the basis for one result graph.

The result graph controller (132) generates the result graphs B (135) from the files (130). The result graph controller (132) is a collection of programs that may operate on the server (112). For a sentence of the file (131), the result graph controller (132) identifies the result nodes (138) and the result edges (140) for the result graph (137).

The result graphs B (135) are generated from the files (130) and includes the result graph (137), which corresponds to the file (131). The result nodes (138) represent nouns and verbs from a sentence of the file (131). The result edges (140) identify semantic relationships between the words represented by the result nodes (138).

The user devices A (102) and B (107) through N (109) are computing systems (further described in FIG. 12A). For example, the user devices A (102) and B (107) through N (109) may be desktop computers, mobile devices, laptop computers, tablet computers, server computers, etc. The user devices A (102) and B (107) through N (109) include hardware components and software components that operate as part of the system (100). The user devices A (102) and B (107) through N (109) communicate with the server (112) to access, manipulate, and view information including information from the graph data (158) and the file data (155). The user devices A (102) and B (107) through N (109) may communicate with the server (112) using standard protocols and file types, which may include hypertext transfer protocol (HTTP), HTTP secure (HTTPS), transmission control protocol (TCP), internet protocol (IP), hypertext markup language (HTML), extensible markup language (XML), etc. The user devices A (102) and B (107) through N (109) respectively include the user applications A (105) and B (108) through N (110).

The user applications A (105) and B (108) through N (110) may each include multiple programs respectively running on the user devices A (102) and B (107) through N (109). The user applications A (105) and B (108) through N (110) may be native applications, web applications, embedded applications, etc. In one embodiment, the user applications A (105) and B (108) through N (110) include web browser programs that display web pages from the server (112). In one embodiment, the user applications A (105) and B (108) through N (110) provide graphical user interfaces that display information stored in the repository (150).

As an example, the user application A (105) may be operated by a user and generate the request (118) to view information related to entities defined in the ontology library (152), described in the file data (155), and graphed in the graph data (158). Corresponding sentences and images from the file data (155) and graphs from the graph data (158) may be received in the response (125) and displayed in a user interface of the user application A (105).

As another example, the user device N (109) may be used by a developer to maintain the software applications hosted by the server (112) and train the machine learning models used by the system (100). Developers may view the data in the repository (150) to correct errors or modify the application served to the users of the system (100).

The repository (150) is a computing system that may include multiple computing devices in accordance with the computing system (1200) and the nodes (1222) and (1224) described below in FIGS. 12A and 12B. The repository (150) may be hosted by a cloud services provider that also hosts the server (112). The cloud services provider may provide hosting, virtualization, and data storage services as well as other cloud services and to operate and control the data, programs, and applications that store and retrieve data from the repository (150). The data in the repository (150) includes the ontology library (152), the file data (155), the model data (157), and the graph data (158).

The ontology library (152) includes information that of the entities and biomedical terms and phrases used by the system (100). Multiple terms and phrases may be used for the same entity. The ontology library (152) defines types of entities. In one embodiment, the types include the types of protein/gene, chemical, cell line, pathway, tissue, cell type, disease, organism, etc. The ontology library (152) may store the information about the entities in a database, structured text files, combinations thereof, etc.

The file data (155) is biomedical information stored in electronic records. The biomedical information describes the entities and corresponding relationships that are defined and stored in the ontology library (152). The file data (155) includes the files (130). Each file in the file data (155) may include image data and text data. The image data includes images that represent the graphical figures from the files. The text data represents the writings in the file data (155). The text data for a file includes multiple sentences that each may include multiple words that each may include multiple characters stored as strings in the repository (150). In one embodiment, the file data (155) includes biomedical information stored as extensible markup language (XML) files, portable document files (PDFs). The file formats define containers for the text and images of the biomedical information describing evidence of biomedical experiments.

The model data (157) includes the data for the models used by the system (100). The models may include rules-based models and machine learning models. The machine learning models may be updated by training, which may be supervised training. The modeling application (128) may load the models from the model data (157) to generate the result graphs B (135) from the files (130).

The model data (157) may also include intermediate data. The intermediate data is data generated by the models during the process of generating the result graphs B (135) from the files (130).

The graph data (158) is the data of the graphs (including the evidence graph (121) and the result graphs A (120) and B (135)) generated by the system. The graph data (158) includes the nodes and edges for the graphs. The graph data (158) may be stored in a database, structured text files, combinations thereof, etc.

Although shown using distributed computing architectures and systems, other architectures and systems may be used. In one embodiment, the server application (115) may be part of a monolithic application that implements evidence networks. In one embodiment, the user applications A (105) and B (108) through N (110) may be part of monolithic applications that implement evidence networks without the server application (115).

Turning to FIG. 1B, the result graph controller (132) processes the file (131) to generate the result graphs B (135). In one embodiment, the result graph controller (132) includes the sentence controller (160), the token controller (162), the tree controller (164), and the text graph controller (167) to process the text from the file (131) describing biomedical evidence. In one embodiment, the result graph controller (132) includes the image controller (170), the text controller (172), and the image graph controller (177) to process the figures from the file (131) that provide evidence for the conclusions of experiments.

The sentence controller (160) is a set of programs that operate to extract the sentences (161) from the file (131). In one embodiment, the sentence controller (160) cleans the text of the file (131) by removing markup language tags, adjusting capitalization, etc. The sentence controller (160) may split a string of text into substrings with each substring being a string that includes a sentence from the original text of the file (131). In one embodiment, the sentence controller (160) may filter the sentences and keep sentences with references to the figures of the file (131).

The sentences (161) are text strings extracted from the file (131). A sentence of the sentences (161) may be stored as a string of text characters. In one embodiment, the sentences (161) are stored in a list that maintains the order of the sentences (161) from the file (131). In one embodiment, the list may be filtered to remove sentences that do not contain a reference to a figure.

The token controller (162) is a set of programs that operate to locate the tokens (163) in the sentences (161). The token controller (162) may identify the start and stop of each token in a sentence

The tokens (163) identify the boundaries of words in the sentences (161). In one embodiment, a token (of the tokens (163)) may be a substring of a sentence (of the sentences (161)). In one embodiment, a token (of the tokens (163)) may be a set of identifiers that identify the locations of a start character and a stop character in a sentence. Each sentence may include multiple tokens.

The tree controller (164) is a set of programs that operate to generate the trees (165) from the tokens (163) of the sentences (161) of the file (131). In one embodiment, the tree controller (164) uses a neural network (e.g., the Berkeley Neural Parser)

The trees (165) are syntax trees of the sentences (161) to identify the parts of speech of the tokens (163) within the sentences (161). In one embodiment, the trees (165) are graphs with edges identifying parent child relationships between the nodes of a graph. In one embodiment, the nodes of a graph of a tree include a root node, intermediate nodes, and leaf nodes. The leaf nodes correspond to tokens (words, terms, multiword terms, etc.) from a sentence and the intermediate nodes identify parts of speech of the leaf nodes.

The text graph controller (167) is a set of programs that operate to generate the result graphs B (135) from the trees (165). In one embodiment, the text graph controller (167) maps the tokens (163) from the sentences (161) that represent nouns and verbs to nodes of the result graphs B (135). In one embodiment, the text graph controller (167) maps parts of speech identified by the trees (165) to the edges of the result graphs B (135).

In one embodiment, after generating an initial graph (of the result graphs B (135)) for a sentence (of the sentences (161)), the text graph controller (167) processes the graph using the ontology library (152) to identify the entities and corresponding entity types represented by the nodes of the graph. For example, a node of the graph may correspond to the token “BRD9”. The text graph controller (167) identifies the token as an entity defined in the ontology library (152) and identifies the entity type as a protein.

The image controller (170) is a set of programs that operate to extract figures from the file (131) to generate the images (171). The image controller also extracts the figure text (169) that corresponds to the images (171). In one embodiment, the image controller (170) may use rules and logic to identify the images and corresponding image text from the file (131). In one embodiment, the image controller (170) may use machine learning models to identify the images (171) and the figure text (169). For example, the file (131) may be stored in a page friendly format (e.g., a portable document file (PDF)) in which each page of a publication is stored as an image in a file. A machine learning model may identify pages that include figures and the locations of the figures on those pages. The located figures may be extracted as the images (171). Another machine learning model may identify the legend text that corresponds to and describes the figures, which is extracted as the figure text (169).

The images (171) are image files extracted from the file (131). In one embodiment, the file (131) includes the figures as individual image files that the image controller (170) converts to the images (171). In one embodiment, the figures of the file (131) may be contained within larger images, e.g., the image of a page of the file (131). The image controller (170) processes the larger images to extract the figures as the images (171).

The figure text (169) is the text from the file (131) that describes the images (171). Each figure of the file (131) may include legend text that describes the figure. The legend text for one or more figures of the file (131) is extracted as the figure text (169), which corresponds to the images (171).

The text controller (172) is a set of programs that operate to process the images (171) and the figure text (169) to generate the structured text (173). The text controller (172) is further described with FIG. 1C below.

The structured text (173) is strings of nested text with information extracted from the images (171) using the figure text (169). In one embodiment, the structured text (173) includes a JSON formatted string for each image of the images (171). In one embodiment, the structured text (173) identifies the locations of text, panels, and experiment metadata within the images (171). In one embodiment, the structured text (173) includes text that is recognized from the images (171). The structured text (173) may include additional metadata about the images (171). For example, the structured text may identify the types of experiments and the types of techniques used in the experiments that are depicted in the images (171).

The image graph controller (177) is a set of programs that operate to process the structured text (173) to generate one or more of the result graphs B (135). In one embodiment, the image graph controller (177) identifies text that corresponds to entities defined in the ontology library (152) from the structured text (173) and maps the identified text to nodes of the result graphs B (135). In one embodiment, the image graph controller (177) uses the nested structure of the structure text (173) to identify the relationships between the nodes of one or more of the result graphs B (135) and maps the relationships to edges of one or more of the result graphs B (135).

The result graphs B (135) are the graphs generated from the file (131) by the result graph controller (132). The result graphs B (135) include nodes that represent entities defined in the ontology library (152) and include edges that represent relationships between the nodes.

The ontology library (152) defines the entities that may be recognized by the result graph controller (132) from the file (131). The entities defined by the ontology library (152) are input to the token controller (162), the text graph controller (167), and the image graph controller (177), which identify the entities within the text and image extracted from the file (131).

Turning to FIG. 1C, the text controller (172) processes the image (180) and the corresponding legend text (179) to generate the image text (188). The text controller (172) may operate as part of the result graph controller (132) of FIG. 1B.

The image (180) is one of the images (171) from FIG. 1B. The image (180) includes a figure from the file (131) of FIG. 1B.

The legend text (179) is a string from the figure text (169) of FIG. 1B. The legend text (179) is the text from the legend of the figure that corresponds to the image (180).

The text detector (181) is a set of programs that operate to process the image (180) to identify the presence and location of text within the image (180). In one embodiment, the text detector (181) uses machine learning models to identify the presence and location of text. The location may be identified with a bounding box that specifies four points of a rectangle that surrounds text that has been identified in the image (180). The location of the text from the text detector (181) may be input to the text recognizer (182).

The text recognizer (182) is a set of programs that operates to process the image (180) to recognize text within the image (180) and output the text as a string. The text recognizer (182) may process a sub image from the image (180) that corresponds to a bounding box identified by the text detector (181). A machine learning model may then be used to recognize the text from the sub image and output a string of characters that correspond to the text within the sub image.

The panel locator (183) is a set of programs that operates to process the image (180) to identify the location of panels and subpanels within the image (180) or a portion of the image (180). A panel of the image (180) is a portion of the image, which may depict evidence of an experiment. The panels of the image (180) may contain subpanels to further subdivide information contained within the image (180). The image (180) may include multiple panels and subpanels that may identified within the legend text (179). The panel locator (183) may be invoked to identify the location for each panel (or subpanel) identified in the legend text (179). In one embodiment, the panel locator (183) outputs a bit array with each bit corresponding to a pixel from the image (180) and identifying whether the pixel corresponds to a panel.

The experiment detector (184); is a set of programs that operates to process the image (180) to identify metadata about experiments depicted in the image (180). In one embodiment, the experiment detector (184) processes the image (180) with a machine learning model (e.g., a convolutional neural network) that outputs a bounding box and a classification. In one embodiment, the bounding box may be an array of coordinates (e.g., top, left, bottom, right) in the image that identify the location of evidence of an experiment within the image. In one embodiment, the classification may be a categorical value that identifies experiment metadata, which may include the type of evidence, the type of experiment, or technique used in the experiment (e.g., graph, western blot, etc.).

The text generator (185) is a set of programs that operate to process the outputs from the text detector (181), the text recognizer (182), the panel locator (183), and the experiment detector (184) to generate the image text (188). In one embodiment, the text generator (185) creates a nested structure for the image text (188) based on the outputs from the panel locator (183), the experiment detector (184), and the text detector (181). For example, the text generator (185) may include descriptions for the panels, experiment metadata, and text from the image (180) in which the text and description of the experiment metadata may be nested within the description of the panels. Elements for subpanels may be nested within the elements for the panels.

The image text (188) is a portion of the structured text (173) of FIG. 1B that corresponds to the image (180). In one embodiment, the image text (188) uses a nested structure to describe the panels, experiment metadata, and text that are identified and located within the image (180).

Turning to FIG. 1D, the evidence graph controller (143) processes the result graphs A (120) to generate the evidence graph (121). The evidence graph controller (143) may operate as part of the interface controller (122) of FIG. 1A.

The evidence graph (121) stores relationships between entities from the ontology library (152) (of FIG. 1A) and corresponding files publications from the file data (155) (of FIG. 1A). The evidence graph (121) includes the evidence nodes (123) and the evidence edges (126).

The evidence nodes (123) (including the evidence node (124)) are the nodes of the evidence graph (121). The evidence nodes (123) represent the entities from the ontology library (152) (of FIG. 1A). For example, the evidence node (124) may correspond to the protein EPAS1.

The evidence edges (126) (including the evidence edge (127)) are the edges of the evidence graph (121). The evidence edges (126) represent an aggregation of the files that link the entities of the nodes of the evidence graph (121). The evidence edges (126) may be identified from the result graphs generated from files of the file data (155). For example, the evidence edge (127) may connect the evidence node (124) with another node of the evidence graph (121). The connection by the evidence edge (127) indicates that at least one file includes at least one result graph in which a path exists between the evidence node (124) and the other node of the evidence edge (127). In one embodiment, the path exists when at least one sentence from the file includes the alias names of the entities represented by the evidence node (124) and the other node. In one embodiment, the evidence graph controller (143) generates the evidence graph (121) in response to instructions from the request (118) (of FIG. 1A).

Turning to FIG. 1E, the file controller (148) presents information from the file (131). In one embodiment, the file controller (148) updates the response (125) (of FIG. 1A) to include the result graph (137), the image (180), and the sentence (159) from the file (131).

The file (131) is a file of biomedical information. The file (131) may correspond to the evidence edge (127) of the evidence graph (121). The file (131) is stored in the file data (155) (of FIG. 1A), includes the sentence (159) (from which the result graph (137) is generated) and includes the image (180), which corresponds to the sentence (159).

The result graph (137) is a result graph generated from the sentence (159) from the file (131). The result graph includes the result nodes (138) (including the result node (139)) and includes the result edges (140) (including the result edge (141)). The result node (139) may correspond to the same entity to which the evidence node (124) corresponds. The result edge (141) may identify a semantic relationship between the result node (139) and another result node from the result graph (137).

The sentence (159) is a sentence from the file (131). The sentence (159) is parsed to generate the result graph (137).

The image (180) is an image from the file (131). The image (180) comprises a figure from the file (131) that is referred to in the sentence (159).

Turning to FIG. 2, the process (200) implements evidence network navigation. The process (200) may be performed by a computing system, such as the computing system (1200) of FIG. 12A.

At Step 202, a user input corresponding to an entity of an ontology library is received. In one embodiment, the user input is provided to a user device by a user. The user device may send the user input to a server that receives the message. In one embodiment, the user input may be received as part of a request. The request may identify one or more entities from the ontology library stored in the repository. In one embodiment, the request may include one or more filters to apply to an evidence graph.

At Step 205, an evidence graph is generated using the user input. In one embodiment, the evidence graph includes an evidence node representing the entity from the ontology library and includes an evidence edge representing a file that includes the entity in a result graph. One evidence node may represent multiple files that each include the entity in a respective result graph. An evidence node connects between two entities of the ontology library. Each file of an evidence node connects the same two entities from the ontology library using a result graph that is specific to (and may be different for) each file. In one embodiment, the result graph includes a result node representing the entity and a result edge representing a semantic relationship of the result node in a sentence from the file.

In one embodiment, the evidence graph may be generated by searching graph data in a repository. The search identifies a set of result graphs (of sentences from files) that include a result node corresponding to the entity from the user input. The entities of the result nodes from the set of result graphs may be used to generate the evidence nodes of the evidence graph. The evidence edges of the evidence graph identify the files from which the set of result graphs were formed.

At Step 208, the evidence graph is presented. The evidence graph may be presented by transmitting the evidence graph to a user device as a response to a request from the user device. The user device may display the evidence graph in a user interface that shows the evidence nodes and evidence edges of the evidence graph. The evidence nodes of the evidence graph may have different colors or shapes that identify the entity types of the evidence nodes. For example, evidence nodes that represent proteins may be displayed as a circle or with the color red and evidence nodes that represent diseases may be shaped with a square or with the color blue, etc.

In one embodiment, the result graph is presented with a sentence and an image from the file. The sentence references the image. The sentence may be the sentence from which the result graph was generated. The result graph may be presented in response to a user input.

In one embodiment, an entity list is presented in response to the user input. The entity list may include an entity name based on the user input and an alias link for the entity name. The entity list includes entity names for the entities with alias names that match the input. For example, the user types in three letters (for a protein) and the system may search the alias names of the entities for a match and then include the entities (with an alias name matching the three letters) in the entity list.

Each item in the entity list corresponds to an entity and each item may include an alias link. Selecting the alias link of an entity item in the entity list may display an alias list of alias names for the entity of the entity item. Selection may be by hovering the mouse over the link, clicking on the link, etc.

In one embodiment, a filter input is received. The filter input may identify an entity type. In one embodiment, the user may select the filter input from a user interface displaying an evidence graph. The filter input may be received by a user interface element. For example, the user interface element may be part of a panel with multiple user interface elements for filtering the evidence graph.

The evidence graph may be filtered using the filter input. The filter input may identify an entity type to remove from the evidence graph.

The evidence graph may be presented in response to filtering the evidence graph. After updating the evidence graph by adding or removing evidence nodes and evidence edges, the evidence graph may be transmitted to the user device that provide the filter input, which may display the evidence graph with the updates.

In one embodiment, a filter input is received that identifies a number of experiments. The number of experiments identifies a number of result graphs corresponding to the entity. A minimum number and a maximum number of experiments may be identified.

The evidence graph is filtered using the number of result graphs. An experiment may correlate to a result graph. The evidence graph may be filtered by removing evidence edges (and corresponding evidence nodes) that have a number of result graphs that are below the minimum number of experiments or are above the maximum number of experiments.

The evidence graph is presented in response to filtering the evidence graph. The evidence graph may be transmitted to and displayed by user device after being filtered by the number of experiments.

In one embodiment, a filter input is received that identifies an entity to remove from the evidence graph. The entity identified may be different from the initial entity used to generate the evidence graph.

The evidence graph is filtered using the entity. For example, the evidence graph may be processed to identify the result graphs of the evidence edges that include the entity to be filtered. The identified result graphs and corresponding evidence edges and evidence nodes may be removed from the evidence graph.

The evidence graph is presented in response to filtering the evidence graph. The evidence graph may be transmitted to and displayed by user device after being filtered by the entity.

In one embodiment, a filter input is received that identifies one of a publication type and a publication year for the biomedical information in a file. The publication types may include “published” for publications that have been printed in a journal and “preprint” for publication that have not been printed in a journal. The publication year may include a minimum year and a maximum year.

The evidence graph is filtered using the filter input. The files, and corresponding evidence edges and evidence nodes, may be removed from the evidence graph based on the filter input.

The evidence graph is presented in response to filtering the evidence graph. The evidence graph may be transmitted to and displayed by user device after being filtered by the publication type and publication year.

In one embodiment, a selection of an evidence node of the evidence graph is received. The selection may be made by clicking on the evidence node of an evidence graph displayed in a user interface of a user device.

The evidence graph may be expanded at the evidence node to include additional evidence nodes connected to the selected evidence node with one or more evidence edges. The expansion searches the graph data for result graphs that include the selected evidence node.

The evidence graph is presented in response to expanding the evidence graph. The evidence graph may be transmitted to and displayed by user device after expanding the selected evidence node.

In one embodiment, a selection of the evidence edge of the evidence graph is received. The selection may be received in response to a user clicking on the evidence edge of the evidence graph displayed in a user interface on a user device.

A file list corresponding to the evidence edge may be presented. A file item of the file list corresponds to the file and includes a color based on a context of the file (e.g., “published” or “preprint”), an identification of the file, an image generated from the file, and a string generated from a sentence of the file.

In one embodiment, a selection of the evidence node of the evidence graph is received. The selection may be received in response to a user clicking on the evidence node of the evidence graph displayed in a user interface on a user device.

An edge list with one or more edge items corresponding to the evidence edge may be presented. The display of an edge item may include the names of the entities of an edge connected to the selected evidence node and a count of the number of experiments represented by the edge. For example, and edge between the HIF1A and VEGFA proteins may include the names of the proteins and include a number (“3198”) that identifies the number of experiments that use both proteins. The number of experiments may be determined by counting the number of result graphs that include a path between nodes representing the entities identified by the edge item.

In one embodiment, the result graph is presented with a sentence and an image from the file. The sentence from the file references the image that is presented with the result graph.

An entity list may be presented that includes the entities from the result graph. The entities in the entity list may be grouped by entity type.

In one embodiment, the evidence graph is presented to a user device. The evidence graph may be presented with a user interface element for sharing the evidence graph.

A graph link may be presented in response to a selection of the user interface element. The graph link identifies a state of the evidence graph, which may include the filters applied to the evidence graph.

A request may be received that includes the graph link and is from a second device. The evidence graph may be presented to the second device.

In one embodiment, an export file is transmitted in response to a selection of an export link. The export file may include a file list that includes multiple files or result graphs of biomedical information in a tabular format. For example, a comma separated value (CSV) file may be used with columns for the name of the publication, type of publication, date of publication, etc. of one or more files. The export link may be an export link for an evidence edge of an evidence graph and the CSV file may include a link or other identifier for the files or publications corresponding to the evidence edge.

In one embodiment, a second user input corresponding to a second entity of the ontology library is received. The second user input may be received using a user interface element for adding an entity to the evidence graph. The user interface element may be displayed a pane, which may be displayed to the left of the evidence graph. The evidence graph may be generated using the user input and the second user input.

The evidence graph may be presented with evidence edges connected to the evidence node representing the first entity or a second node representing the second entity. In one embodiment, the evidence graph may be presented with evidence edges connected to the evidence node representing the first entity and to the second node representing the second entity. In one embodiment, the evidence graph may be presented with evidence edges in which multiple paths between the evidence node representing the first entity and to a second node representing the second entity. Each path may start or end with the evidence node or the second node. In one embodiment, the evidence graph may be presented with evidence edges evidence edges connected to one of the evidence node representing the first entity and the second node representing the second entity.

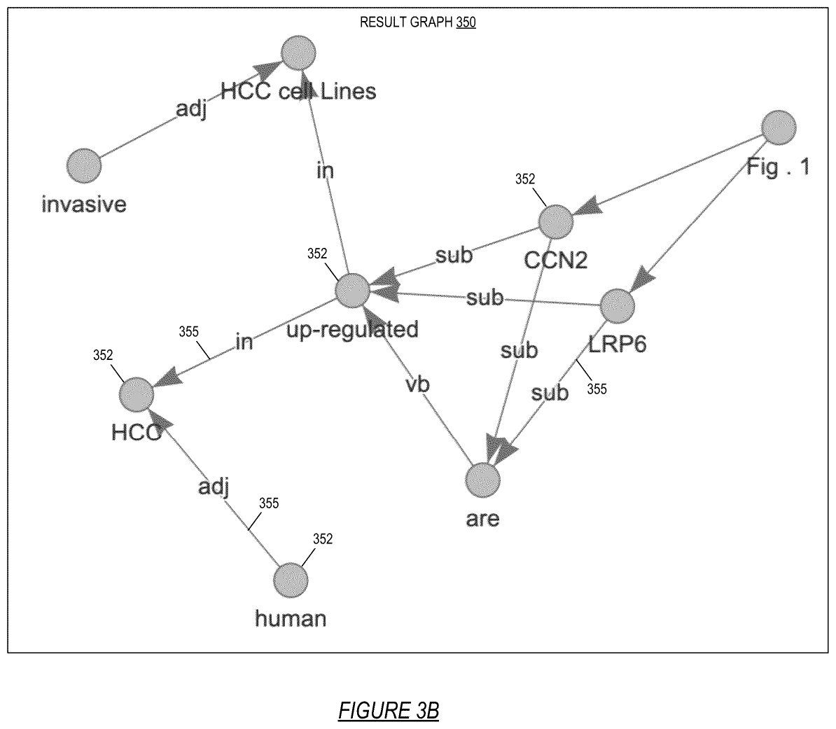

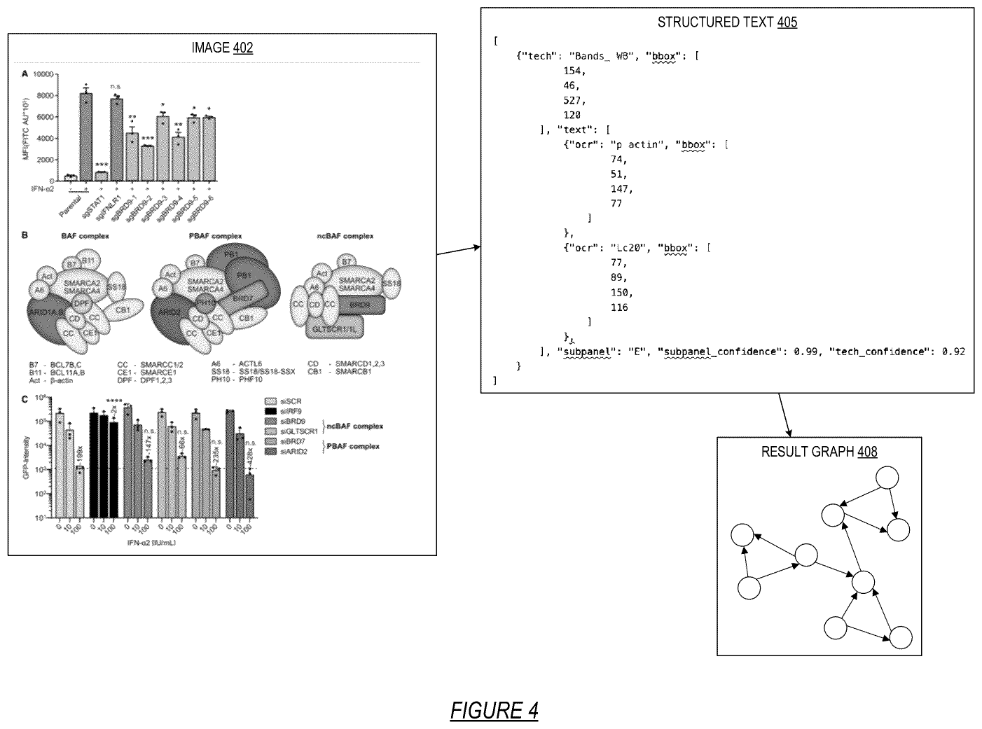

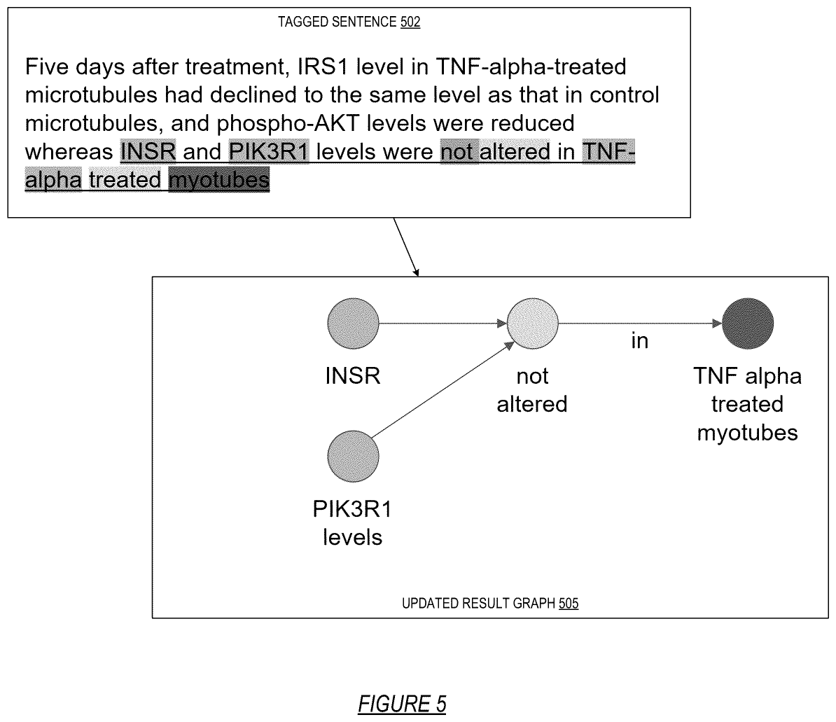

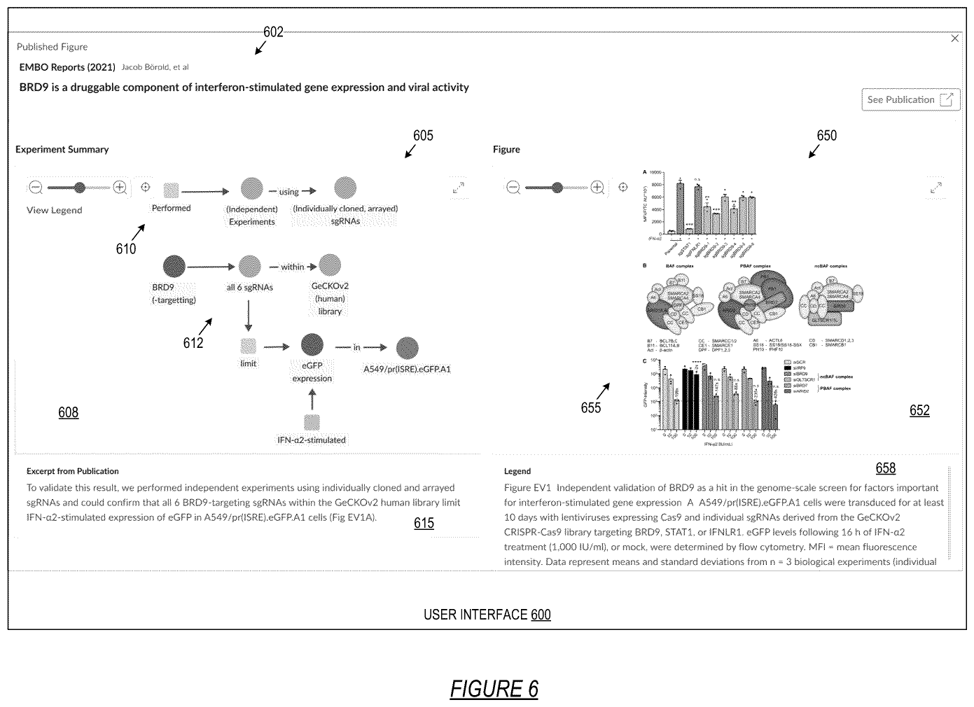

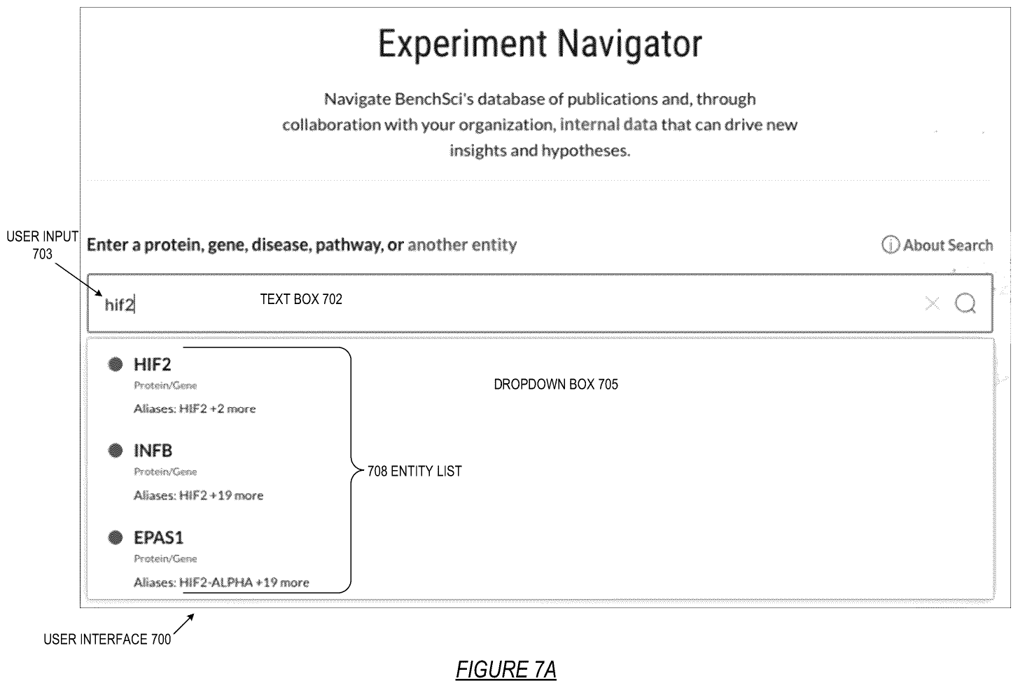

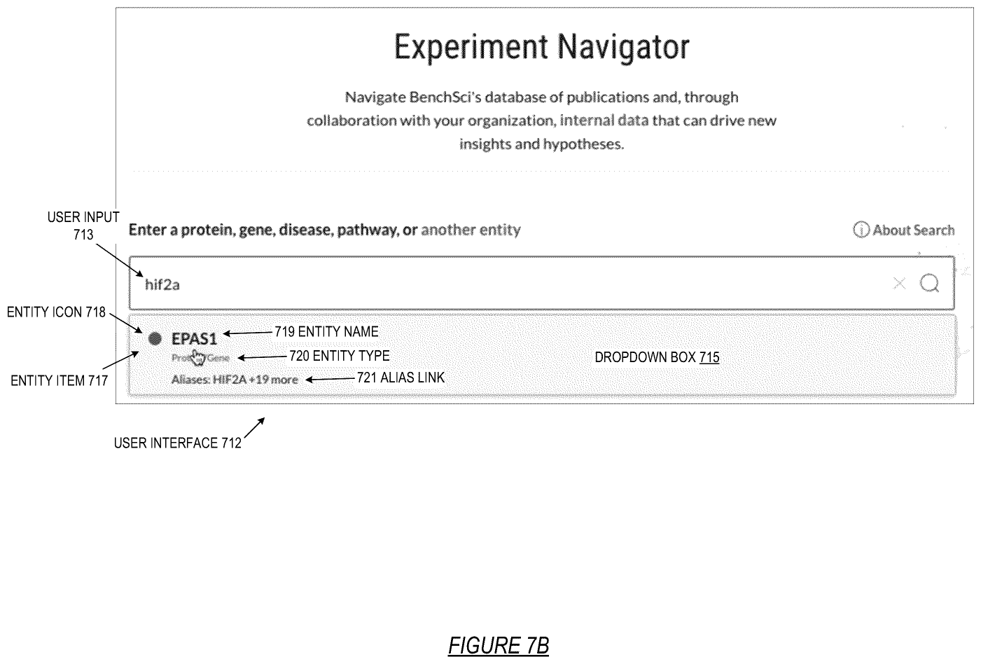

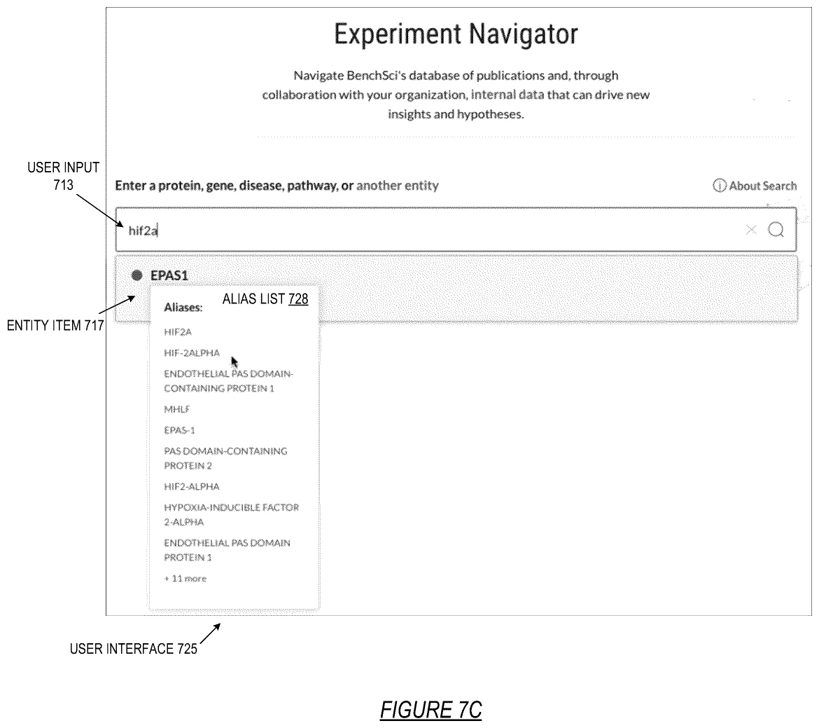

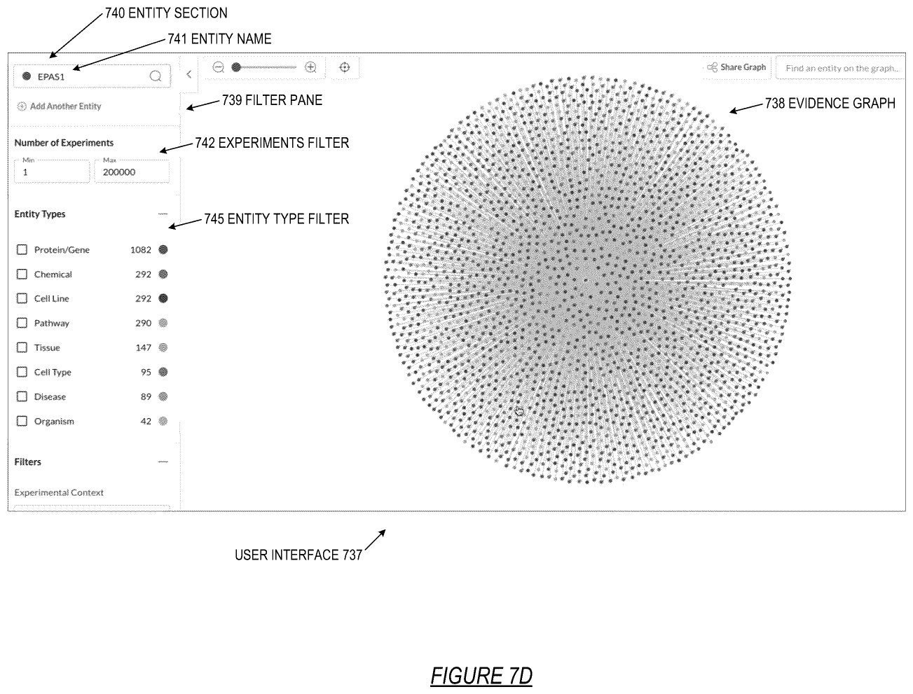

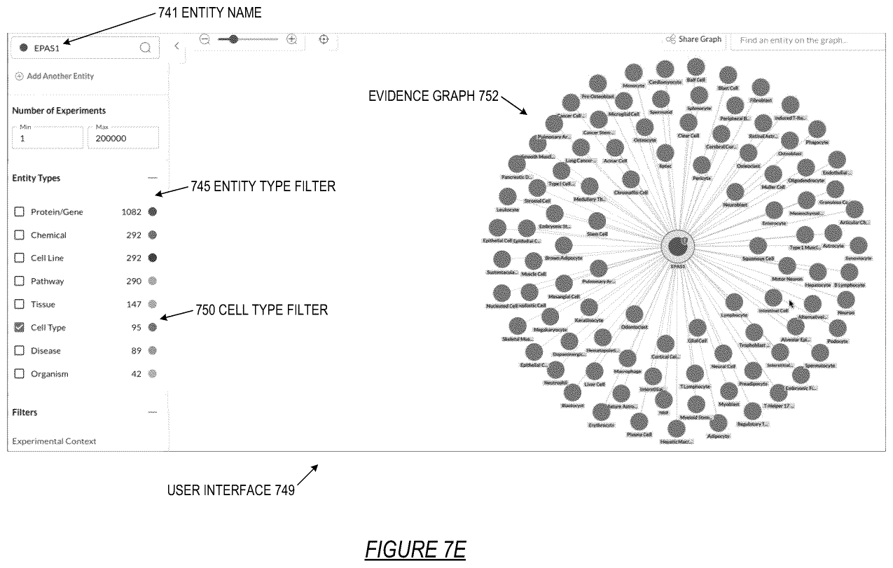

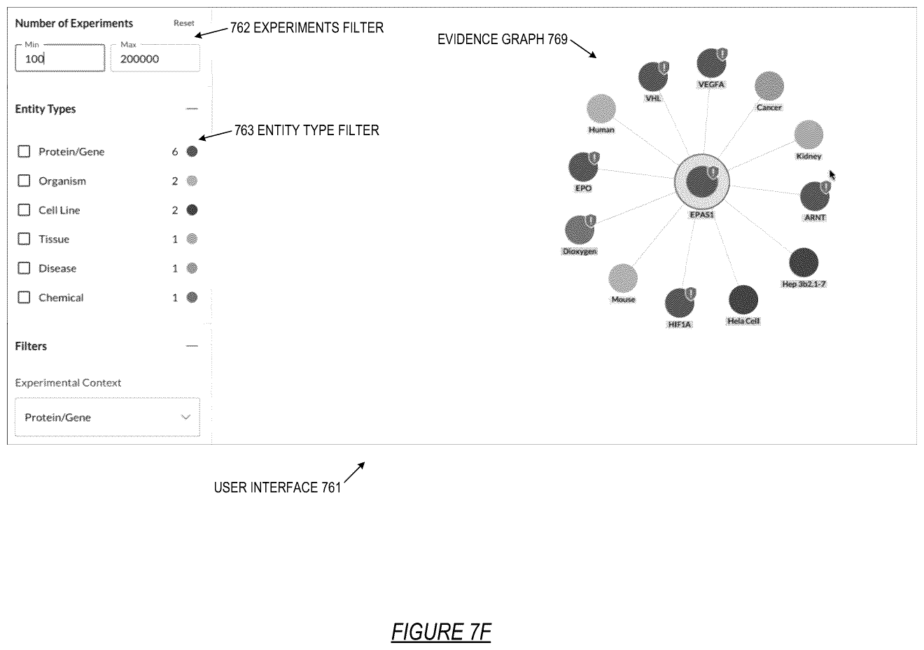

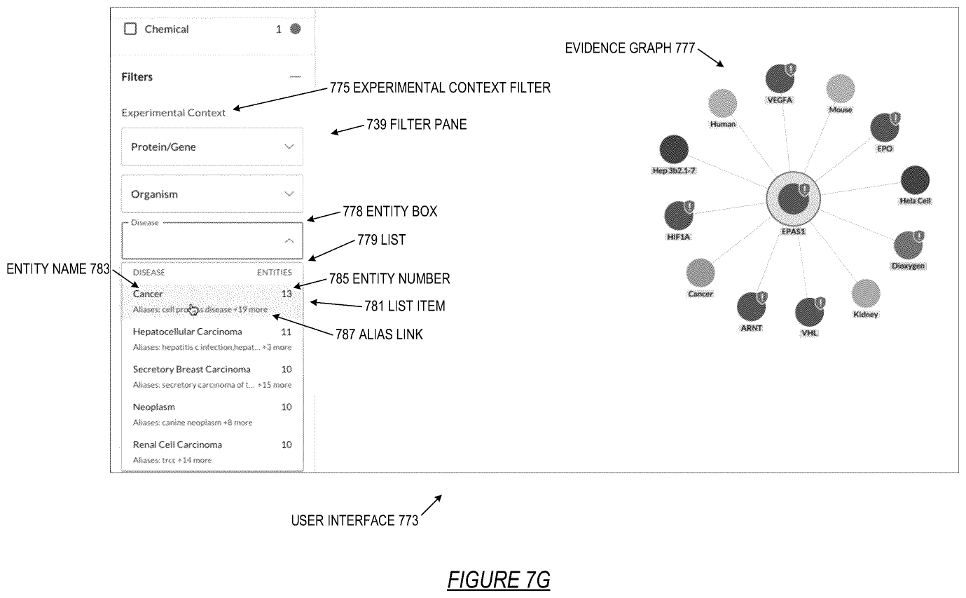

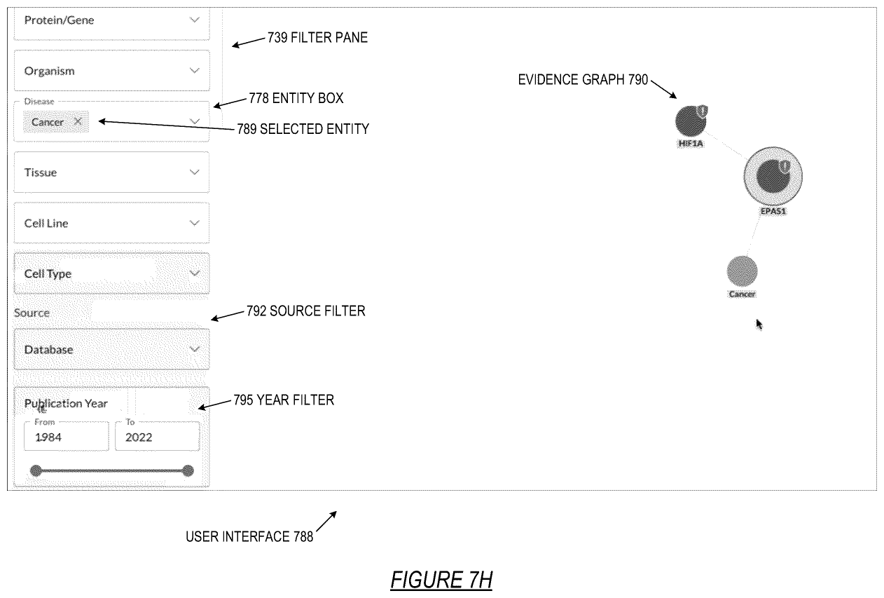

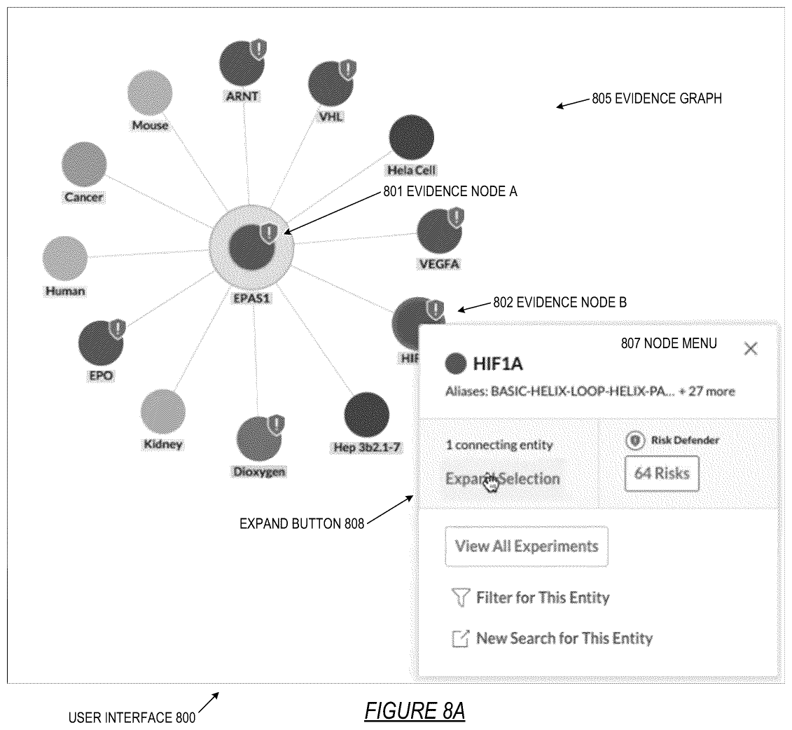

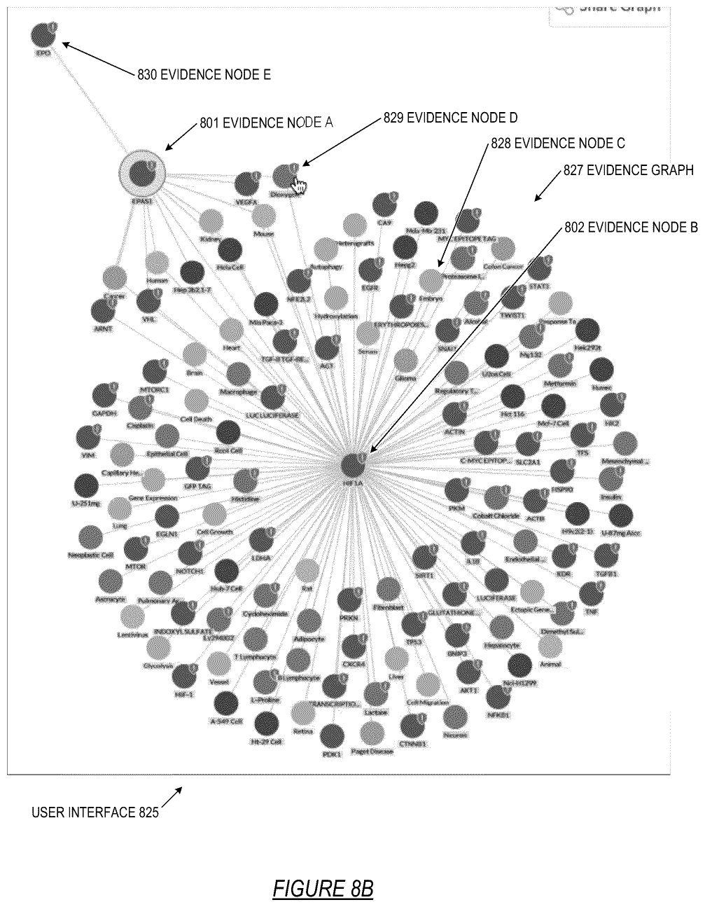

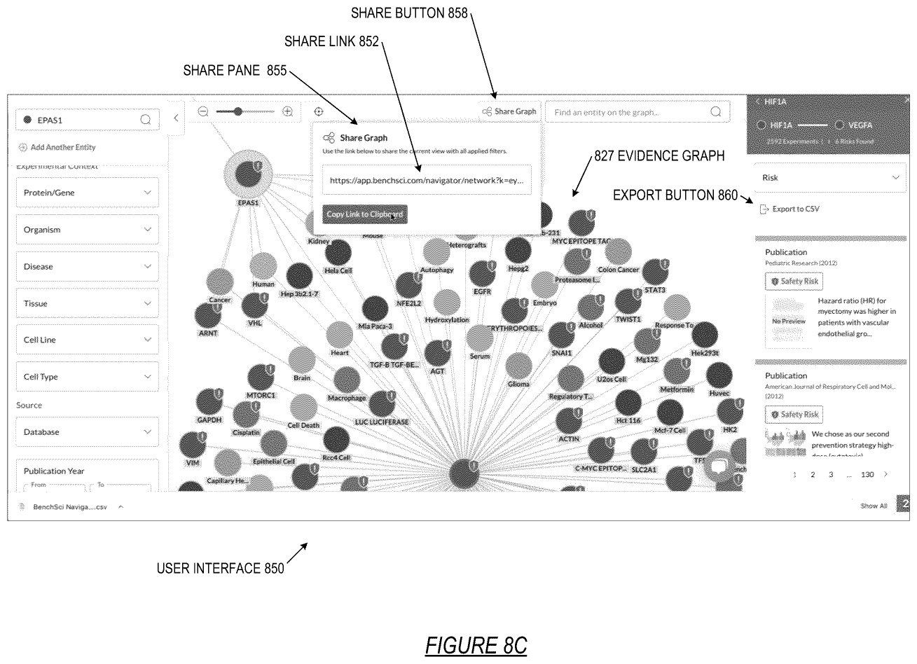

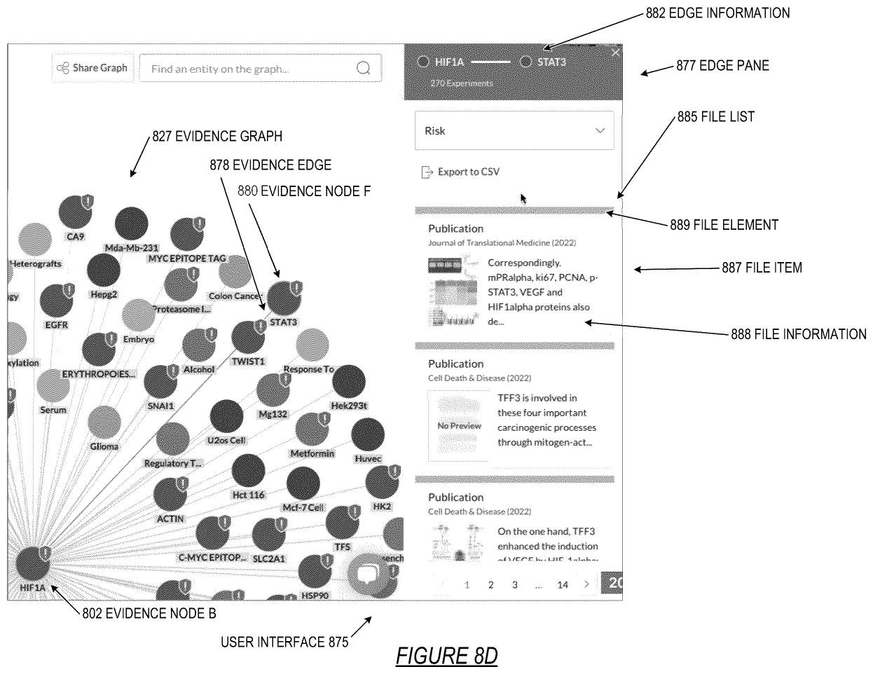

Turning to FIG. 3A, the file (302) is shown from which the sentence (305) is extracted, which is used to generate the tree (308), which is used to generate the graph (350) (of FIG. 3B). The file (302), the sentence (305), the tree (308), and the result graph (350) (of FIG. 3B) may be stored as electronic files, transmitted in messages, and displayed on a user interface.

The file (302) is a collection of biomedical information, which may include, but is not limited to, a writing of biomedical literature with sentences and figures stored as text and images. Different sources of biomedical information may be used. The file (302) is processed to extract the sentence (305).

The sentence (305) is a sentence from the file (302). The sentence (305) is stored as a string of characters. In one embodiment, the sentence (305) is tokenized to identify the locations of entities within the sentence (305). For example, the entities recognized from the sentence (305) may include “CCN2”, “LRP6”, “HCC”, and “HCC cell lines”. The sentence (305) is processed to generate the tree (308).

The tree (308) is a data structure that identifies semantic relationships of the words of the sentence (305). The tree (308) includes the leaf nodes (312), the intermediate nodes (315), and the root node (318).

The leaf nodes (312) correspond to the words from the sentence (305). The leaf nodes have no child nodes. The leaf nodes have parent nodes in the intermediate nodes (315).

The intermediate nodes (315) include values that identify the parts of speech of the leaf nodes (312). The intermediate nodes (315) having leaf nodes as direct children nodes identify the parts of speech of the words represented by the leaf nodes. The intermediate nodes (315) that do not have leaf nodes as direct children nodes identify the parts of speech of groups of one or more words, i.e., phrases, of the sentence (305).

The root node (318) is the top of the tree (308). The root node (318) has no parent node.

Turning to FIG. 3B, the result graph (350) is a data structure that represents the sentence (305) (of FIG. 3A). The result graph (350) may be generated from the sentence (305) and the tree (308). The nodes of the result graph (350) represent nouns (e.g., “CCN2”, “HCC”, etc.) and verbs (e.g., “up-regulated”, “are”, etc.) from the sentence (305). The edges (355) identify semantic relationships (e.g., subject “sub”, verb “vb”, adjective “adj”) between the words of the nodes (352) of the sentence (305) (of FIG. 3A). The result graph (350) is a directed acyclic graph.

Turning to FIG. 4, the image (402) is shown from which the structured text (405) is generated, which is used to generate the result graph (408). The image (402), the structured text (405), and the result graph (408) may be stored as electronic files, transmitted in messages, and displayed on a user interface.

The image (402) is a figure from a file (e.g., the file (302) of FIG. 3A, which may be from a biomedical publication). In one embodiment, the image (402) is an image file that is included with or as part of the file (302) of FIG. 3A. In one embodiment, the image (402) is extracted from an image of a page of a publication stored as the file (302) of FIG. 3A. The image (402) includes three panels labeled “A”, “B”, and “C”. The “B” panel includes three subpanels labeled “BAF complex”, “PBAF complex”, and “ncBAF complex”. The image (402) is processed to recognize the locations of the panels, subpanels, and text using machine learning models. After being located, the text from the image is recognized and stored as text (i.e., strings of characters). The panel, subpanel, and text locations along with the recognized text are processed to generate the structured text (405).

The structured text (405) is a string of text characters that represents the image (402). In one embodiment, the structured text (405) includes nested lists that form a hierarchical structure patterned after the hierarchical structure of the panels, subpanels, and text from the image (402). The structured text (405) is processed to generate the result graph (408).

The result graph (408) is a data structure that represents the figure, corresponding to the image (402), from a file (e.g., the file (302) of FIG. 3A). The result graph (408) includes nodes and edges. The nodes represent nouns and verbs identified in the structured text (405). The edges may represent the nested relationships between the panels, subpanels, and text of the image (402) described in the structured text (405).

Turning to FIG. 5, the tagged sentence (502) is generated from a sentence and used to generate the updated result graph (505). The tagged sentence (502) and the updated result graph (505) may be stored as electronic files, transmitted in messages, and displayed on a user interface.

The tagged sentence (502) is a sentence from a file that has been processed to generate the updated result graph (505). The sentence from which the tagged sentence is derived is input to a model to tag the entities in the sentence to generate the tagged sentence (502). The model may be a rules-based model, an artificial intelligence model, combinations thereof, etc.

As an example, the underlined portion (“INSR and PIK3R1 levels were not altered in TNF-alpha treated myotubes”) is tagged by the model. The terms “INSR”, “PIK3R1”, and “TNF-alpha” may be tagged as one type of entity that is presented as green when displayed on a user interface. The term “not” is tagged and may be displayed as orange. The terms “altered” and “treated” are tagged and may be displayed as pink. The term “myotubes” is tagged and may be displayed as red. After being identified in the sentence, the tags may be applied to the graph to generate the updated result graph (505).

The updated result graph (505) is an updated version of a graph of the sentence used to generate the tagged sentence (502). The graph is updated to label the nodes of the graph with the tags from the tagged sentence. For example, the nodes corresponding to “INSR” and “PIK3R1” are labeled with tags identified in the tagged sentence and may be displayed as green. The node corresponding to “altered” is tagged and displayed as pink. The node corresponding to “myotubes” is tagged and displayed as red.

Turning to FIG. 6, the user interface (600) displays information from a file, which may be a publication of biomedical literature. Different sources of files may be used. The user interface (600) may display the information on a user device after receiving a response to a request for the information transmitted to a server application. For example, the request may be for a publication that includes evidence linking the proteins “BRD9” and “A549”. The user interface displays the header section (602), the summary section (605), and the figure section (650).

The header section (602) includes text identifying the file being displayed. In one embodiment, the text in the header section (602) includes the name of the publication, the name of the author, the title of the publication, etc., which may be extracted from the file. Additional sources of information may be used, including patents, ELN data, summary documents, portfolio documents, scientific data in raw/table form, presentations, etc., and similar information may be extracted.

The summary section (605) displays information from the text of the file identified in the header section (602). The summary section (605) includes the graph section (608) and the excerpt section (615).

The graph section (608) includes the result graphs (610) and (612). the result graphs (610) and (612) were generated from the sentence displayed in the excerpt section (615). The result graph (612) shows the link between the proteins “BRD9” and “A549”, which conforms to the request that prompted the response with the information displayed in the user interface (600).

The excerpt section (615) displays a sentence from the file identified in the header section (602). The sentence in the excerpt section (615) is the basis from which the result graphs (610) and (612) were generated by tokenizing the sentence, generating a tree from the tokens, and generating the result graphs (610) and (612) from the tokens and tree.

The figure section (650) displays information from the figures of the file identified in the header section (602). The figure section (650) includes the image section (652) and the legend section (658).

The image section (652) displays the image (655). The image (655) was extracted from the file identified in the header section (602). The image (655) corresponds to the text from the legend section (658). The image (655) corresponds to the result graph (612) because the sentence shown in the excerpt section (615) identifies the figure (“Fig EV1A”) that corresponds to the image (655).

The legend section (658) displays the text of the legend that corresponds to the figure of the image (655). In one embodiment, the text of the legend section (655) may be processed to generate one or more graphs from the sentence in the legend section (658).

FIGS. 7A through 7H, 8A through 8D, 9A through 9C, 10A through 10B, and 11A through 11G illustrate user interfaces that display evidence graphs. In one embodiment, the evidence graphs may be generated by a server and displayed on a user device.

Turning to FIG. 7A, the user interface (700) displays several user interface elements to identify an entity from which to create an evidence graph. The user interface (700) includes the text box (702) and the dropdown box (705).

The text box (702) is a user interface element that receives text input by the user. The text box (702) includes the user input (703).

The user input (703) is text entered by the user to the text box (702). The system compares the text from the user input (703) to the alias names of the entities of an ontology library. Entities that include alias names that at least partially match to the text from user input (703) or populated into the entity list (708) of the dropdown box (705).

The dropdown box (705) is displayed in response to the user input (703). The dropdown box (705) includes the entity list (708).

The entity list (708) includes a list of entity items. The entity items correspond to entities, of an ontology library, that include an alias name that matches to the text from the user input (703).

Turning to FIG. 7B, the user input (713) is updated to identify an alias name of a specific entity. The dropdown box (715) is updated to include an entity list with the single item, the entity item (717).

The entity item (717) is the single entity item displayed in the dropdown box (715) in response to the user input (713). The entity item (717) includes the entity icon (718), the entity name (719), the entity type (720), and the alias link (721).

The entity icon (718) is an image that corresponds to the entity of the entity item (717). In one embodiment, the color of the entity icon (718) is determined from the entity type (720). For example, when the entity of the entity item (717) is for a protein or gene, the entity icon (718) may be displayed with the color blue. Different colors may be used for different entity types.

The entity name (719) is the name of the entity to which the entity item (717) corresponds. The entity name (719) may be a primary name for the entity, which may include multiple alias names.

The entity type (720) identifies the entity type of the entity to which the entity item (717) corresponds. The entity types may include proteins, genes, diseases, experiment techniques, chemicals, cell lines, pathways, tissues, cell types, organisms, etc.

The alias link (721) provides information about the aliases of the entity corresponding to the entity item (717). The alias link (721) may display the alias that matches to the user input (713). The alias link (721) may further identify the number of alias names for the entity of the entity item (717).

Turning to FIG. 7C, the user interface (725) is updated from the user interface (712) of FIG. 7B. In response to selection of the alias link (721) (of FIG. 7B), the alias list (728) is displayed.

The alias list (728) displays multiple aliases for the entity of the entity item (717). The alias list (728) may display a subset of alias names that are assorted by the similarity to the user input. The similarity may be determined by calculating a Dice coefficient, a Levenshtein distance, etc., between the alias name and the user input.

Turning to FIG. 7D, the user interface (737) is updated from the user interface (725) of FIG. 7C. The user interface (737) is updated to display the evidence graph (738) and the filter pane (739).

The evidence graph (738) is generated from the entity identified by the user input (713) of FIG. 7B. The evidence graph (738) includes multiple evidence nodes and evidence edges. The evidence nodes represent entities from the ontology library. The evidence edges represent the connections between the evidence nodes. An evidence edge between two evidence nodes indicates that there is a file with evidence that includes both of the entities identified by the nodes of the edge. The evidence is described in a sentence of the file from which a result graph is generated. The result graph includes a path between two result nodes that represent the same entities as the evidence nodes of the evidence edge. In one embodiment, the evidence node at the center of the evidence graph (738) is the evidence node that represents the entity that corresponds to the entity name (741), which corresponds to the entity identified by the user input (713) of FIG. 7C.

The filter pane (739) is a user interface element that includes additional user interface elements for filtering the evidence graph (738). The additional user interface elements are responsive to user interaction to filter the evidence nodes and the evidence edges of the evidence graph (738). The filter pane (739) includes the entity section (740), the experiments filter (742), and the entity type filter (745).

The entity section (740) includes a text box. The text box includes the entity name (741), which identifies the entity of the evidence node at the center of the evidence graph (738). The text box provides suggestions and aliases like the text box (702) of FIG. 7A. User input to the text box of the entity section (740) to change the entity name (741) may trigger a rebuild of the evidence graph (738).

The experiments filter (742) filters the evidence graph (738) based on the number of experiments per evidence edge between the evidence node. In one embodiment, the number of experiments corresponds to the number of files that include references to the entities of the nodes of an edge. The experiments filter (742) may receive inputs for a minimum number of experiments and a maximum number of experiments. In order for an edge to be displayed on the evidence graph (738), the edge may include a number of experiments that is between (inconclusively) the minimum number of experience and the maximum number of experiments. In one embodiment, the number of experiments between two evidence nodes is the number of sentences (from the files analyzed by the system) that include the names of the entities that correspond to the evidence nodes. In one embodiment, the number of sentences may be determined by identifying the number of result graphs that include paths between result nodes that correspond to these same entities as the evidence nodes.

The entity type filter (745) is a collection of user interface elements used to specify the entity types for filtering the evidence graph (738). The entity type filter (745) may include a check box element for each type of entity to include with or remove from the evidence graph (738). The user interface elements for an entity type include a check box, the name of the entity type, the number of evidence nodes of the evidence graph (738) that corresponds to the entity type, and a color for the entity type. The different entity types may have different colors or shapes when displayed in the evidence graph (738) and identified in the entity type filter (745).

Turning to FIG. 7E, the user interface (749) is updated from the user interface (737) of FIG. 7D. The user interface (749) is updated based on a selection of the cell type filter (750) from the entity type filter (745).

The cell type filter (750) is selected to filter the evidence graph (752). The evidence graph (752) is filtered to show a central evidence node for the entity identified by the entity name (741) (“EPAS1”) and surrounding evidence nodes that represent entities that are cell types. By selecting the cell type filter (750), the number of surrounding nodes is reduced to the central evidence node, the 95 surrounding evidence nodes, and corresponding evidence edges. Each evidence edge indicates the existence of a file with a sentence from which result graph is generated that includes result nodes that represent the same entities as the evidence nodes of the evidence edge.

Turning to FIG. 7F, the user interface (761) is updated from the user interface (749) of FIG. 7E. The user interface (761) is updated based interaction with the experiments filter (762).

The experiments filter (762) is updated after receiving a user input to set the minimum number of experiments to “100” (from “1”). In response to the user input, the evidence graph (769) is updated as well as the entity type filter (763) being reset.

The evidence graph (769) is updated so that edges with a minimum number of “100” experiments (and a maximum number of “200,000”) are displayed. Increasing the minimum number of experiments reduces the number of evidence edges and of corresponding evidence nodes of the evidence graph (769) as compared to the evidence graph (752) of FIG. 7E.

The entity type filter (763) is updated by being reset and removing the cell type filter (750) that was selected in FIG. 7E. The cell type filter (750) (of FIG. 7E) is removed from the entity type filter (763) because the evidence graph (769) does not include surrounding evidence knowns that correspond to entities that are cell types. After removing the cell type filter (750), the antitype filter is reset to show the entity types that are present in the evidence graph (769).

Turning to FIG. 7G, the user interface (773) is updated from the user interface (761) of FIG. 7F. The user interface (773) is updated based on interaction with the experimental context filter (775) of the filter pane (739).

The experimental context filter (775) is displayed after scrolling down in the filter pane (739). The experimental context filter (775) filters the evidence graph (777) for specific entities identified in the experimental context filter (775). The experimental context filter (775) includes entity boxes (e.g., the entity box (778)) for each type of entity in the surrounding evidence nodes of the evidence graph (777).

The entity box (778) is for filtering for diseases. Selecting the entity box (778) displays the list (779). The list (779) includes list items (e.g., the list item (781)) for each of the entities that are diseases and are present in a result graph (generated from a file) corresponding to an evidence edge of the evidence graph (777).

The list item (781) corresponds to an entity defined in the ontology library. The list item (781) corresponds to “cancer” as identified by the entity name (783). The entity number (785) indicates that “13” result graphs, corresponding to the evidence edges of the evidence graph (777), include a result node corresponding to “cancer”. The alias link (787) displays and indicates that additional alias names may identify the entity corresponding to the entity name (783) (“cancer”). Selection of the alias link (787) may bring up a list of the additional alias names.

Turning to FIG. 7H, the user interface (788) is updated from the user interface (773) of FIG. 7G. The user interface (788) is updated after selection of the selected entity (789).

The selected entity (789) is selected using the entity box (778) of the filter pane (739). The entity box (778) is for entities that are of the “disease” entity type. The entity box (778) may be used to select multiple entities (e.g., multiple “diseases”).

The evidence graph (790) is updated in response to the selection of the selected entity (789). The evidence graph (790) is updated to remove the evidence edges and evidence nodes that do not correspond to a result graph that includes the entity identified by the selected entity (789).

The source filter (792) is included in the filter pane (739). The source filter (792) may filter the evidence graph (790) by the source of the files used to generate the result graphs that are used to generate the evidence graph (790). For example, the source filter (792) may filter by “publication” or by “preprint”. The “publication” filter removes files for publications that have not been published in a peer reviewed journal from the evidence edges of the evidence graph (790). The “preprint” filter removes files for publications that have been published in a peer reviewed journal from the evidence edges of the evidence graph (790).

The year filter (795) is included in the filter pane (739). The year filter (795) may filter the evidence graph (790) by the year of the publications used to generate the result graphs that are used to generate the evidence graph (790). For example, the year filter (795) may filter by removing files of publications with dates older than a minimum (“from”) date and removing files of publications with dates younger than a maximum (“to”) date. The files are removed from the evidence edges of the evidence graph (790).

Turning to FIG. 8A, the user interface (800) is updated. The user interface (800) is updated after selection of the evidence node B (802).

The evidence node B (802) is one of the evidence nodes of the evidence graph (805). The evidence node B (802) identifies an entity (named “HIF1A”) for which a corresponding result node was located in a result graph (generated from a sentence of a file) that also includes then entity identified by the evidence node A (801). Selection of the evidence node B (802) brings up the node menu (807).

The node menu (807) displays multiple user interface elements exposing information and functions. The node menu (807) includes the name of the entity represented by the evidence node B (802) and includes an alias link that identifies multiple alias for the entity. The node menu (807) also includes the expand button (808).

The expand button (808) is a user interface element displayed on the node menu (807). Selection of the expand button will expand the evidence graph (805) to include evidence nodes and evidence edges for result graphs (and corresponding files) that include result nodes that correspond to the entity identified by the evidence node B (802) (e.g., “HIF1A”).

Turning to FIG. 8B, the user interface (825) is updated from the user interface (800) of FIG. 8A. The user interface (825) is updated to display the evidence graph (827) after selection of the expand button (808) of FIG. 8A.

The evidence graph (827) is updated from the evidence graph (805) of FIG. 8A. The evidence graph (827) is updated to include additional evidence nodes (e.g., the evidence node C (828)) (and evidence edges) corresponding to the evidence node B (802).

The evidence nodes A (801) and B (802) of the evidence graph (827) form the basis from which the evidence graph (827) is generated. Each of the evidence edges in the evidence graph (827) indicate that at least one file includes evidence using one or both of the entities identified by the evidence nodes A (801) and B (802). The evidence edges are identified from the result graphs generated from the files analyzed by the system. An evidence edge of the evidence graph (827) indicates that a sentence of a file includes an alias name of either of the entities identified by the evidence nodes A (801) and B (802). For example, sentences that include the terms “EPO” and “HIF1A” are used to generate result graphs that include result nodes for the entities “EPO” and “HIF1A”. The result graphs are located in the graph data. Additional result nodes and corresponding evidence are identified from the result graphs located in the graph data. For example, the evidence node C (828) identifies the entity “embryo”, which was found in at least one sentence (of a file) from which a result graph was generated that also included the entity “HIF1A”.

The evidence graph (827) includes primary nodes (e.g., the evidence nodes A (801) and B (802)), which form the basis of the search to generate the evidence graph (827), and includes secondary nodes (e.g., the evidence nodes C (828), D (829), and E (830)), which are the evidence nodes found when searching the graph data for the entities of the primary nodes.

The secondary nodes relate to one or more of the primary nodes. For example, the evidence node C (828) relates to, and shares an edge with, the evidence node B (802), but does not share an edge with the evidence node A (801). Sharing an edge indicates that a result graph was generated from a sentence (of a file) that included of the entities identified by the evidence nodes of the shared edge. The evidence node D (829) shares edges with both the evidence nodes A (801) and B (802). The evidence node E (830) shares an edge with the evidence node A (801) but not with the evidence node B (802).

Turning to FIG. 8C, the user interface (850) is updated. The user interface (850) is updated to display the share link (852).

The share link (852) is displayed in the share pane (855), which is displayed in response to selection of the share button (858). The share link (852) is a uniform resource locator (URL) that may be accessed with a web browser by another user device. For example, a user may copy the share link (852) to an email sent to another user. The other user may paste the share link (852) into a browser to view the evidence graph (827). The evidence graph (827) is viewed the same between both devices and includes the selections to the filters used to generate the evidence graph (827). In one embodiment, the share link (852) may include the information used to generate the evidence graph (827) within the text of the share link (852). The information used to generate the evidence graph (827) may be coded. In one embodiment, the share link includes a unique identifier that the server uses to locate the evidence graph (827).

The export button (860) is included in the user interface (850). Selection of the export button (860) may bring up a pane for exporting information from the evidence graph (827) to a file. For example, the information from the evidence graph (827) may be exported to a tabular text file (e.g., a comma separated value (CSV) file) with rows for each of the files that are identified from the graph. A row may include the information about the file and about the graph. For example, a row may identify the name of the file and identify the names of the entities that correspond to the evidence nodes of an evidence edge of evidence graph (827).

Turning to FIG. 8D, the user interface (875) is updated. The user interface (875) is updated to display the edge pane (877).

The edge pane (877) is displayed upon selection of the evidence edge (878). The evidence edge (878) connects between the evidence nodes B (802) and F (880). The edge pane (877) displays the edge information (882) and the file list (885).

The edge information (882) describes the evidence edge (878). The edge information (882) identifies the names of the entities for the evidence nodes B (802) (“HIF1A”) and F (880) (“STAT3”). The edge information (882) indicates that “(270)” experiments include the entities identified as “HIF1A” and “STAT3”. An experiment may be identified as a sentence from a file of biomedical information that includes the names (or aliases) for the entities identified as “HIF1A” and “STAT3”.

The file list (885) includes a list of file items, which includes the file item (887). A file item may be included for each of the experiments enumerated in the edge information (882).

The file item (887) describes a file that corresponds to the evidence edge (878). The file item (887) includes the file information (888) and the file element (889).

The file information (888) display information from the file of the file item (887). The information may include the title of the publication of the file, the date published, a sentence from the file, and an image from the file. The sentence is the sentence from which a result graph was generated that was identified when searching the graph data to generate the evidence graph (827). The image is a figure from the file that is referenced in the sentence used to generate the result graph that was identified when searching the graph data to generate the evidence graph (827). The sentence and image may be previews of the sentence and image from the file. The preview of the sentence may be a shortened version of the sentence and may include the name of the entity from a primary node (e.g., the evidence node B (802)). The preview of an image may be a smaller version with lower resolution of the image of the figure from the file.

The file element (889) may be displayed above the file information (888). The file element (889) may include a color that identifies the type of publication of the file. For example, one color may indicate that the file has been published and a different color may indicate that the file is a preprint version that has not been published.

Turning to FIG. 9A, the user interface (900) is updated. The user interface (900) is updated to display the primary node menu (902).

The primary node menu (902) includes user interface elements that display information about the evidence node B (802) and expose functionality. The information about the evidence node B (802) identifies the name of the entity (“HIF1A”), identifies the number of alias names, and displays some of the alias names. The primary node menu (902) includes the view experiments button (905).

The view experiments button (905) is a user interface element displayed in the primary node menu (902). Selecting the view experiments button (905) displays the experiments related to the evidence node B (802).

Turning to FIG. 9B, the user interface (933) is updated from the user interface (900) of FIG. 9A. The user interface (933) is updated to display the primary node pane (935).

The primary node pane (935) is displayed in response to selection of the view experiments button (905) of FIG. 9A. The primary node pane (935) includes the primary node information (937) and the edge list (938).

The primary node information (937) displays information about the evidence node B (802) of the evidence graph (827). The primary node information (937) displays the name of the entity (“HIF1A”) and identifies the number (“(124)”) of evidence edges connected to the evidence node B (802).

The edge list (938) is a list of items for the evidence edges connected to the evidence node B (802). The edge list (938) includes the edge item (941).

The edge item (941) describes an evidence edge. The edge item (941) includes names (“HIF1A” and “VEGFA”) for the entities of the evidence nodes of the evidence edge. The edge item (941) identifies the number of experiments (“(3198)”) that correspond to the evidence edge that corresponds to the edge item (941). Selecting the edge item (941) may bring up an edge pane.

Turning to FIG. 9C, the user interface (966) is updated from the user interface (933) of FIG. 9B. The user interface (966) is updated to display the edge pane (968).

The edge pane (968) displays the file list (970). The file list (970) is a user interface element used to display the file that correspond to the evidence edge of the edge pane (968). The file list (970) includes the file item (972). Selection of the file item (972) brings up a file pane. The file item (972) includes the sentence preview (975) and the image preview (978).

Turning to FIG. 10A, the user interface (1000) is updated. The user interface (1000) is updated to display the file pane (1002).

The file pane (1002) is displayed in response to selection of the file item (972) (of FIG. 9C). The file pane (1002) displays the file information (1005), the result graph (1008), the sentence (1010), the image (1012), and the legend (1015).

The file information (1005) describes the file from which the result graph (1008) is generated. The file information (1005) includes the name of the file, the date of the publication of the file, and the title of the article from the file.

The result graph (1008) is displayed in the file pane (1002). The result graph (1008) is generated from the sentence (1010). The sentence (1010) is the sentence from which the sentence preview (975) (of FIG. 9C) was extracted.

The image (1012) is displayed in the file pane (1002). The image (1012) corresponds to the sentence (1010) and is referenced in the text of the sentence (1010). The legend (1015) is the text that describes the image (1012).

Turning to FIG. 10B, the user interface (1050) is updated from the user interface (1000) of FIG. 10A. The user interface (1050) is updated to display an additional portion of the file pane (1002).

The file pane (1002) includes the entities used button (1052). When the entities used button (1052) is selected, the entity list (1055) is displayed.

The entity list (1055) is displayed in the file pane (1002). The entity list (1055) displays the entities identified by the result nodes from the result graph (1008). The entities of the entity list (1055) are grouped by entity type.

Turning to FIG. 11A, the user interface (1100) is updated. The user interface (1100) is updated to display the evidence graph (1102). The evidence graph (1102) is generated by selecting the entity (1105) (“EPAS1”) as the primary entity, setting the minimum number of experiments to “10”, and filtering the entity types, using the entity type filter (1107), to “protein/gene”. Another primary entity may be added to the evidence graph (1102) using the add entity button (1108).

Turning to FIG. 11B, the user interface (1115) is updated from the user interface (1100) of FIG. 11A. The user interface (1115) is updated to display the edit box (1118) after selection of the add entity button (1108) from FIG. 11A.

Turning to FIG. 11C, the user interface (1128) is updated from the user interface (1115) of FIG. 11B. The user interface (1128) is updated to display the entity list (1110) with the entity item (1112).

Turning to FIG. 11D, the user interface (1142) is updated from the user interface (1128) of FIG. 11C. The user interface (1142) is updated to display the evidence graph (1145) and the graph type selector (1153).

The evidence graph (1145) is updated from the evidence graph (1102) of FIG. 11A. The evidence graph (1145) is updated to show the entity (1105) (“EPAS1”) and the second entity (1147) (“Pulmonary Arterial Hyp . . . ”) as the evidence nodes A (1149) and B (1150). The evidence nodes evidence nodes A (1149) and B (1150) are displayed as primary nodes with an extra circle around image of the node.

The evidence graph (1145) includes a set of evidence nodes (including the evidence node C (1151)) that are connected to the evidence node A (1149) but for which there is no path to the evidence node B (1150). The evidence graph (1145) includes a set of evidence nodes (including the node D (1152)) that are connected to the evidence node A (1149) and for which there exists a path through the evidence graph (1145) between the evidence nodes A (1149) and B (1150).

The graph type selector (1153) is a user interface element of the user interface (1142). The graph type selector (1153) includes the graph type A (1155). Selection the graph type A (1155) displays the evidence graph (1145), which displays the evidence nodes that are connected by an evidence edge to one or more of the evidence nodes A (1149) and B (1150).

Turning to FIG. 11E, the user interface (1156) is updated from the user interface (1142) of FIG. 11D. The user interface (1156) is updated to display the evidence graph (1157) using the graph type B (1158).

The evidence graph (1157) is updated from the evidence graph (1145) of FIG. 11D using the graph type B (1158), which shows the “common” files between the entities represented by the evidence nodes A (1149) and B (1150). The evidence graph (1157) includes the evidence edge (1159) between the evidence nodes A (1149) and B (1150). The evidence edge (1159) represents the files that include sentences with both of the entities represented by the evidence nodes A (1149) and B (1150).

Turning to FIG. 11F, the user interface (1170) is updated from the user interface (1156) of FIG. 11E. The user interface (1170) is updated to display the evidence graph (1171) using the graph type C (1172).

The evidence graph (1171) is updated from the evidence graph (1157) of FIG. 11E using the graph type C (1172), which shows the “shared” files between the entities represented by the evidence nodes A (1149) and B (1150). The evidence graph (1171) includes the evidence edges that share connections between the evidence nodes A (1149) and B (1150).

Turning to FIG. 11G, the user interface (1185) is updated from the user interface (1170) of FIG. 11F. The user interface (1185) is updated to display the evidence graph (1187) using the graph type D (1188).

The evidence graph (1187) is updated from the evidence graph (1171) of FIG. 11F using the graph type D (1188), which shows the “unique” files that connect to the evidence nodes A (1149) and B (1150). The evidence graph (1187) includes the evidence edges that do not share connections between the evidence nodes A (1149) and B (1150).

Embodiments of the invention may be implemented on a computing system. Any combination of a mobile, a desktop, a server, a router, a switch, an embedded device, or other types of hardware may be used. For example, as shown in FIG. 12A, the computing system (1200) may include one or more computer processor(s) (1202), non-persistent storage (1204) (e.g., volatile memory, such as a random access memory (RAM), cache memory), persistent storage (1206) (e.g., a hard disk, an optical drive such as a compact disk (CD) drive or a digital versatile disk (DVD) drive, a flash memory, etc.), a communication interface (1212) (e.g., Bluetooth interface, infrared interface, network interface, optical interface, etc.), and numerous other elements and functionalities.

The computer processor(s) (1202) may be an integrated circuit for processing instructions. For example, the computer processor(s) (1202) may be one or more cores or micro-cores of a processor. The computing system (1200) may also include one or more input device(s) (1210), such as a touchscreen, a keyboard, a mouse, a microphone, a touchpad, an electronic pen, or any other type of input device.

The communication interface (1212) may include an integrated circuit for connecting the computing system (1200) to a network (not shown) (e.g., a local area network (LAN), a wide area network (WAN) such as the Internet, a mobile network, or any other type of network) and/or to another device, such as another computing device.

Further, the computing system (1200) may include one or more output device(s) (1208), such as a screen (e.g., a liquid crystal display (LCD), a plasma display, a touchscreen, a cathode ray tube (CRT) monitor, a projector, or other display device), a printer, an external storage, or any other output device. One or more of the output device(s) (1208) may be the same or different from the input device(s) (1210). The input and output device(s) (1210 and (1208)) may be locally or remotely connected to the computer processor(s) (1202), non-persistent storage (1204), and persistent storage (1206). Many different types of computing systems exist, and the aforementioned input and output device(s) (1210 and (1208)) may take other forms.

Software instructions in the form of computer readable program code to perform embodiments of the invention may be stored, in whole or in part, temporarily or permanently, on a non-transitory computer readable medium such as a CD, a DVD, a storage device, a diskette, a tape, flash memory, physical memory, or any other computer readable storage medium. Specifically, the software instructions may correspond to computer readable program code that, when executed by a processor(s), is configured to perform one or more embodiments of the invention.

The computing system (1200) in FIG. 12A may be connected to or be a part of a network. For example, as shown in FIG. 12B, the network (1220) may include multiple nodes (e.g., node X (1222), node Y (1224)). Each node may correspond to a computing system, such as the computing system (1200) shown in FIG. 12A, or a group of nodes combined may correspond to the computing system (1200) shown in FIG. 12A. By way of an example, embodiments of the invention may be implemented on a node of a distributed system that is connected to other nodes. By way of another example, embodiments of the invention may be implemented on a distributed computing system having multiple nodes, where each portion of the invention may be located on a different node within the distributed computing system. Further, one or more elements of the aforementioned computing system (1200) may be located at a remote location and connected to the other elements over a network.

Although not shown in FIG. 12B, the node may correspond to a blade in a server chassis that is connected to other nodes via a backplane. By way of another example, the node may correspond to a server in a data center. By way of another example, the node may correspond to a computer processor or micro-core of a computer processor with shared memory and/or resources.

The nodes (e.g., node X (1222), node Y (1224)) in the network (1220) may be configured to provide services for a client device (1226). For example, the nodes may be part of a cloud computing system. The nodes may include functionality to receive requests from the client device (1226) and transmit responses to the client device (1226). The client device (1226) may be a computing system, such as the computing system (1200) shown in FIG. 12A. Further, the client device (1226) may include and/or perform all or a portion of one or more embodiments of the invention.

The computing system (1200) or group of computing systems described in FIGS. 12A and 12B may include functionality to perform a variety of operations disclosed herein. For example, the computing system(s) may perform communication between processes on the same or different system. A variety of mechanisms, employing some form of active or passive communication, may facilitate the exchange of data between processes on the same device. Examples representative of these inter-process communications include, but are not limited to, the implementation of a file, a signal, a socket, a message queue, a pipeline, a semaphore, shared memory, message passing, and a memory-mapped file. Further details pertaining to a couple of these non-limiting examples are provided below.

Based on the client-server networking model, sockets may serve as interfaces or communication channel end-points enabling bidirectional data transfer between processes on the same device. Foremost, following the client-server networking model, a server process (e.g., a process that provides data) may create a first socket object. Next, the server process binds the first socket object, thereby associating the first socket object with a unique name and/or address. After creating and binding the first socket object, the server process then waits and listens for incoming connection requests from one or more client processes (e.g., processes that seek data). At this point, when a client process wishes to obtain data from a server process, the client process starts by creating a second socket object. The client process then proceeds to generate a connection request that includes at least the second socket object and the unique name and/or address associated with the first socket object. The client process then transmits the connection request to the server process. Depending on availability, the server process may accept the connection request, establishing a communication channel with the client process, or the server process, busy in handling other operations, may queue the connection request in a buffer until server process is ready. An established connection informs the client process that communications may commence. In response, the client process may generate a data request specifying the data that the client process wishes to obtain. The data request is subsequently transmitted to the server process. Upon receiving the data request, the server process analyzes the request and gathers the requested data. Finally, the server process then generates a reply including at least the requested data and transmits the reply to the client process. The data may be transferred, more commonly, as datagrams or a stream of characters (e.g., bytes).

Shared memory refers to the allocation of virtual memory space in order to substantiate a mechanism for which data may be communicated and/or accessed by multiple processes. In implementing shared memory, an initializing process first creates a shareable segment in persistent or non-persistent storage. Post creation, the initializing process then mounts the shareable segment, subsequently mapping the shareable segment into the address space associated with the initializing process. Following the mounting, the initializing process proceeds to identify and grant access permission to one or more authorized processes that may also write and read data to and from the shareable segment. Changes made to the data in the shareable segment by one process may immediately affect other processes, which are also linked to the shareable segment. Further, when one of the authorized processes accesses the shareable segment, the shareable segment maps to the address space of that authorized process. Often, only one authorized process may mount the shareable segment, other than the initializing process, at any given time.

Other techniques may be used to share data, such as the various data sharing techniques described in the present application, between processes without departing from the scope of the invention. The processes may be part of the same or different application and may execute on the same or different computing system.

Rather than or in addition to sharing data between processes, the computing system performing one or more embodiments of the invention may include functionality to receive data from a user. For example, in one or more embodiments, a user may submit data via a graphical user interface (GUI) on the user device. Data may be submitted via the graphical user interface by a user selecting one or more graphical user interface widgets or inserting text and other data into graphical user interface widgets using a touchpad, a keyboard, a mouse, or any other input device. In response to selecting a particular item, information regarding the particular item may be obtained from persistent or non-persistent storage by the computer processor. Upon selection of the item by the user, the contents of the obtained data regarding the particular item may be displayed on the user device in response to the user's selection.

By way of another example, a request to obtain data regarding the particular item may be sent to a server operatively connected to the user device through a network. For example, the user may select a uniform resource locator (URL) link within a web client of the user device, thereby initiating a Hypertext Transfer Protocol (HTTP) or other protocol request being sent to the network host associated with the URL. In response to the request, the server may extract the data regarding the particular selected item and send the data to the device that initiated the request. Once the user device has received the data regarding the particular item, the contents of the received data regarding the particular item may be displayed on the user device in response to the user's selection. Further to the above example, the data received from the server after selecting the URL link may provide a web page in Hyper Text Markup Language (HTML) that may be rendered by the web client and displayed on the user device.

Once data is obtained, such as by using techniques described above or from storage, the computing system, in performing one or more embodiments of the invention, may extract one or more data items from the obtained data. For example, the extraction may be performed as follows by the computing system (1200) in FIG. 12A. First, the organizing pattern (e.g., grammar, schema, layout) of the data is determined, which may be based on one or more of the following: position (e.g., bit or column position, Nth token in a data stream, etc.), attribute (where the attribute is associated with one or more values), or a hierarchical/tree structure (consisting of layers of nodes at different levels of detail-such as in nested packet headers or nested document sections). Then, the raw, unprocessed stream of data symbols is parsed, in the context of the organizing pattern, into a stream (or layered structure) of tokens (where each token may have an associated token “type”).

Next, extraction criteria are used to extract one or more data items from the token stream or structure, where the extraction criteria are processed according to the organizing pattern to extract one or more tokens (or nodes from a layered structure). For position-based data, the token(s) at the position(s) identified by the extraction criteria are extracted. For attribute/value-based data, the token(s) and/or node(s) associated with the attribute(s) satisfying the extraction criteria are extracted. For hierarchical/layered data, the token(s) associated with the node(s) matching the extraction criteria are extracted. The extraction criteria may be as simple as an identifier string or may be a query presented to a structured data repository (where the data repository may be organized according to a database schema or data format, such as XML).

The extracted data may be used for further processing by the computing system. For example, the computing system (1200) of FIG. 12A, while performing one or more embodiments of the invention, may perform data comparison. Data comparison may be used to compare two or more data values (e.g., A, B). For example, one or more embodiments may determine whether A>B, A=B, A!=B, A<B, etc. The comparison may be performed by submitting A, B, and an opcode specifying an operation related to the comparison into an arithmetic logic unit (ALU) (i.e., circuitry that performs arithmetic and/or bitwise logical operations on the two data values). The ALU outputs the numerical result of the operation and/or one or more status flags related to the numerical result. For example, the status flags may indicate whether the numerical result is a positive number, a negative number, zero, etc. By selecting the proper opcode and then reading the numerical results and/or status flags, the comparison may be executed. For example, in order to determine if A>B, B may be subtracted from A (i.e., A−B), and the status flags may be read to determine if the result is positive (i.e., if A>B, then A−B>0). In one or more embodiments, B may be considered a threshold, and A is deemed to satisfy the threshold if A=B or if A>B, as determined using the ALU. In one or more embodiments of the invention, A and B may be vectors, and comparing A with B requires comparing the first element of vector A with the first element of vector B, the second element of vector A with the second element of vector B, etc. In one or more embodiments, if A and B are strings, the binary values of the strings may be compared.

The computing system (1200) in FIG. 12A may implement and/or be connected to a data repository. For example, one type of data repository is a database. A database is a collection of information configured for ease of data retrieval, modification, re-organization, and deletion. A Database Management System (DBMS) is a software application that provides an interface for users to define, create, query, update, or administer databases.

The user, or software application, may submit a statement or query into the DBMS. Then the DBMS interprets the statement. The statement may be a select statement to request information, update statement, create statement, delete statement, etc. Moreover, the statement may include parameters that specify data, or data container (database, table, record, column, view, etc.), identifier(s), conditions (comparison operators), functions (e.g., join, full join, count, average, etc.), sort (e.g., ascending, descending), or others. The DBMS may execute the statement. For example, the DBMS may access a memory buffer, a reference or index a file for read, write, deletion, or any combination thereof, for responding to the statement. The DBMS may load the data from persistent or non-persistent storage and perform computations to respond to the query. The DBMS may return the result(s) to the user or software application.

The computing system (1200) of FIG. 12A may include functionality to present raw and/or processed data, such as results of comparisons and other processing. For example, presenting data may be accomplished through various presenting methods. Specifically, data may be presented through a user interface provided by a computing device. The user interface may include a GUI that displays information on a display device, such as a computer monitor or a touchscreen on a handheld computer device. The GUI may include various GUI widgets that organize what data is shown as well as how data is presented to a user. Furthermore, the GUI may present data directly to the user, e.g., data presented as actual data values through text, or rendered by the computing device into a visual representation of the data, such as through visualizing a data model.

For example, a GUI may first obtain a notification from a software application requesting that a particular data object be presented within the GUI. Next, the GUI may determine a data object type associated with the particular data object, e.g., by obtaining data from a data attribute within the data object that identifies the data object type. Then, the GUI may determine any rules designated for displaying that data object type, e.g., rules specified by a software framework for a data object class or according to any local parameters defined by the GUI for presenting that data object type. Finally, the GUI may obtain data values from the particular data object and render a visual representation of the data values within a display device according to the designated rules for that data object type.

Data may also be presented through various audio methods. In particular, data may be rendered into an audio format and presented as sound through one or more speakers operably connected to a computing device.

Data may also be presented to a user through haptic methods. For example, haptic methods may include vibrations or other physical signals generated by the computing system. For example, data may be presented to a user using a vibration generated by a handheld computer device with a predefined duration and intensity of the vibration to communicate the data.

The above description of functions presents only a few examples of functions performed by the computing system (1200) of FIG. 12A and the nodes (e.g., node X (1222), node Y (1224)) and/or client device (1226) in FIG. 12B. Other functions may be performed using one or more embodiments of the invention.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A method comprising:
receiving a user input corresponding to an entity of an ontology library;
generating an evidence graph using the user input,
wherein the evidence graph includes an evidence node representing the entity from the ontology library and includes an evidence edge representing a file that includes the entity in a result graph, and
wherein the result graph includes a result node representing the entity and a result edge representing a semantic relationship of the result node in a sentence from the file; and
presenting the evidence graph.

2. The method of claim 1, further comprising:
presenting the result graph with the sentence and an image from the file, wherein the sentence references the image.

3. The method of claim 1, further comprising:
presenting an entity list in response to the user input, wherein the entity list comprises an entity name based on the user input and an alias link for the entity name; and
presenting an alias list in response to selection of the alias link.

4. The method of claim 1, further comprising:
receiving a filter input, wherein the filter input identifies an entity type;
filtering the evidence graph using the filter input; and
presenting the evidence graph in response to filtering the evidence graph.

5. The method of claim 1, further comprising:
receiving a filter input, wherein the filter input identifies a number of experiments, wherein the number of experiments identifies a number of result graphs corresponding to the entity;
filtering the evidence graph using the number of result graphs; and
presenting the evidence graph in response to filtering the evidence graph.

6. The method of claim 1, wherein the entity is a first entity and the method further comprises:
receiving a filter input, wherein the filter input identifies a second entity to remove from the evidence graph;
filtering the evidence graph using the second entity; and
presenting the evidence graph in response to filtering the evidence graph.

7. The method of claim 1, further comprising:
receiving a filter input, wherein the filter input identifies one of a publication type and a publication year;
filtering the evidence graph using the filter input; and
presenting the evidence graph in response to filtering the evidence graph.

8. The method of claim 1, further comprising:
receiving a selection of a second node of the evidence graph;
expanding the evidence graph at the second node to include a plurality of evidence nodes connected to the second node with a plurality of evidence edges; and
presenting the evidence graph in response to expanding the evidence graph.

9. The method of claim 1, further comprising:
receiving a selection of the evidence edge of the evidence graph; and
presenting a file list corresponding to the evidence edge, wherein a file item of the file list corresponds to the file and comprises a color based on a context of the file, an identification of the file, an image generated from the file, and a string generated from a sentence of the file.

10. The method of claim 1, further comprising:
receiving a selection of the evidence node of the evidence graph; and
presenting an edge list comprising an edge item corresponding to the evidence edge.

11. The method of claim 1, further comprising:
presenting the result graph with the sentence and an image from the file, wherein the sentence references the image; and
presenting an entity list comprising a plurality of entities from the result graph and grouped by entity type.

12. The method of claim 1, further comprising:
presenting the evidence graph to a user device.
presenting a graph link in response to a selection, wherein the graph link identifies a state of the evidence graph;
receiving a request comprising the graph link from a second device; and
presenting the evidence graph to the second device.

13. The method of claim 1, further comprising:
transmitting an export file in response to a selection of an export link, wherein the export file comprises a file list comprising the file, in a tabular format.

14. The method of claim 1, wherein the user input is a first user input, the entity is a first entity, the evidence edge is a first evidence edge, and the method further comprises:
receiving a second user input corresponding to a second entity of the ontology library;
generating the evidence graph using the user input and the second user input; and
presenting the evidence graph with a plurality of evidence edges connected to one or more of the evidence node representing the first entity and a second node representing the second entity.

15. The method of claim 1, wherein the user input is a first user input, the entity is a first entity, the evidence edge is a first evidence edge, and the method further comprises:
receiving a second user input corresponding to a second entity of the ontology library;
generating the evidence graph using the user input and the second user input; and
presenting the evidence graph with a plurality of evidence edges connected to the evidence node representing the first entity and to a second node representing the second entity.

16. The method of claim 1, wherein the user input is a first user input, the entity is a first entity, the evidence edge is a first evidence edge, and the method further comprises:
receiving a second user input corresponding to a second entity of the ontology library;
generating the evidence graph using the user input and the second user input; and
presenting the evidence graph with a plurality of evidence edges comprised by a plurality of paths between the evidence node representing the first entity and to a second node representing the second entity.

17. The method of claim 1, wherein the user input is a first user input, the entity is a first entity, the evidence edge is a first evidence edge, and the method further comprises:
receiving a second user input corresponding to a second entity of the ontology library;
generating the evidence graph using the user input and the second user input; and
presenting the evidence graph with a plurality of evidence edges connected to one of the evidence node representing the first entity and a second node representing the second entity.

18. A system comprising:
an evidence graph controller configured to generate an evidence graph; and
an application executing on one or more servers and configured for:
receiving a user input corresponding to an entity of an ontology library, generating the evidence graph using the user input,
wherein the evidence graph includes an evidence node representing the entity from the ontology library and includes an evidence edge representing a file that includes the entity in a result graph, and
wherein the result graph includes a result node representing the entity and a result edge representing a semantic relationship of the result node in a sentence from the file, and
presenting the evidence graph.

19. The system of claim 18, wherein the application is further configured for:
presenting the result graph with the sentence and an image from the file, wherein the sentence references the image.

20. A method comprising:
transmitting a request; and
displaying an evidence graph received in a response to the request,
wherein the evidence graph includes an evidence node representing an entity from an ontology library and includes an evidence edge representing a file that includes the entity in a result graph, and
wherein the result graph includes a result node representing the entity and a result edge representing a semantic relationship of the result node in a sentence from the file.
